(19) United States
(12) Patent Application Publication
(10) Pub. No.: US 2026/0284103 A1
(43) Pub. Date: Sep. 24, 2026

(54) RELB CELLS AND METHODS FOR IMMUNOTHERAPY

(71) Applicant: La Jolla Institute for Immunology, La Jolla, CA (US)

(72) Inventors: Wade Zhang, La Jolla, CA (US); Anjana Rao, La Jolla, CA (US); Patrick Hogan, La Jolla, CA (US)

(73) Assignee: La Jolla Institute for Immunology, La Jolla, CA (US)

(21) Appl. No.: 19/135,311

(22) PCT Filed: Dec. 6, 2023
(86) PCT No.: PCT/US2023/082789

§ 371 Date: Jun. 3, 2025

Related U.S. Application Data
(60) Provisional application No. 63/430,601, filed on Dec. 6, 2022.

(51) Int. Cl. A61K 35/14; A61K 40/31; A61K 40/42; A61P 35/04; C07K 14/705; C07K 16/28; C12N 5/078; C12N 15/63

(52) U.S. Cl. CPC A61K 35/14; A61K 40/31; A61K 40/4211; A61P 35/04; C07K 14/705; C07K 16/2803; C07K 16/2863; C12N 5/0634; C12N 15/63; C07K 2317/21; C07K 2317/24; C07K 2317/53; C07K 2317/565; C07K 2317/622; C07K 2319/03; C12N 2510/00

(57) ABSTRACT

Provided herein is an engineered immune cell modified to decrease expression, function, or both expression and function of any one or more of RelB in the immune cell, as well as methods of making and using same. The immune cell can also express a receptor or ligand that binds at least one tumor antigen or at least one antigen expressed by a pathogen. The cells can be formulated into compositions. The cells and compositions are useful as anti-cancer or ant-tumor therapies, or to treat a pathogenic infection.

### CROSS-REFERENCE TO RELATED PATENT APPLICATION

This application is a U.S. National Phase Application under 35 U.S.C. § 371 of International Application No. PCT/US2023/082789, filed on Dec. 6, 2023, which claims the benefit under 35 U.S.C. § 119(e) of U.S. Provisional Application No. 63/430,601, filed Dec. 6, 2022, the entire content of which is incorporated herein by reference.

### STATEMENT AS TO FEDERALLY SPONSORED RESEARCH

This invention was made with government support under National Institute of Health (NIH) Grant Nos. AI040127, AI109842, and U01 DE028227. The government has certain rights in the invention.

### SEQUENCE LISTING

The instant application contains a Sequence Listing which has been submitted electronically in XML format and is hereby incorporated by reference in its entirety. Said XML copy, created on Apr. 16, 2026, is named 116639-2611_SL.xml and is 37,814 bytes in size.

### TECHNICAL FIELD

The disclosure relates to therapeutic applications of adoptive cell therapies, specifically a method of improving CAR T cell therapies, by preventing or reversing T-cell exhaustion or enhancing T-cell proliferation, for treatment of cancer and chronic infections.

### SUMMARY OF THE DISCLOSURE

Provided herein is an immune cell engineered to decrease expression, function, or both expression and function of V-Rel Avian Reticuloendotheliosis Viral Oncogene Homolog B (RelB) in the immune cell. In another aspect, provided herein is an immune cell engineered to decrease expression and/or function of T cell exhaustion proteins in the engineered immune cell.

In yet another aspect, an immune cell engineered to decrease expression, function, or both expression and function of RelB in the immune cell and/or function of T cell exhaustion proteins in the engineered immune cell is disclosed. Non-limiting examples of the T cell exhaustion protein is Nra4a and/or Tox.

In some embodiments, the engineered immune cell expresses a receptor or ligand that binds at least one tumor antigen or at least one antigen expressed by a pathogen. Non-liming examples of tumor antigens are CD19, mesothelin, ROR1, and/or EGFRvIII.

In yet further embodiments, the engineered immune cell comprises a chimeric antigen receptor (CAR), and optionally expresses a receptor or ligand that binds at least one tumor antigen or at least one antigen expressed by a pathogen.

In yet another aspect, there is provided a method of producing an engineered immune cell, the method comprising decreasing expression, function or both expression and function of RelB in the engineered immune cell.

In yet another aspect, there is provided a method comprising, consisting of, or consisting essentially of decreasing expression and/or function of an isoform of RelB and decreasing expression and/or function of T cell exhaustion proteins in the engineered immune cell.

In yet another aspect, there is provided an engineered immune cell prepared by the methods disclosed herein.

A composition is provided that comprises, or consists essentially of, or yet further consists of, a carrier and any one of the engineered immune cells as disclosed herein.

A kit also is provided that comprises, or consists essentially of, or yet further consists of, compositions, such as the engineered immune cells, polynucleotides and/or vectors for the manufacture of any one of the cells disclosed herein. In a further aspect, instructions are provided for the making and/or use thereof.

In yet another aspect, there is provided a method for stimulating a cell-mediated immune response, the method comprising, or consisting essentially of, or yet further consisting of, contacting a target cell population or tissue containing the immune cell that mediates the immune response with any one of the engineered immune cells as disclosed herein.

In yet another aspect, there is provided a method for inhibiting the growth of a tumor or cancer cell, or inhibiting metastasis of a tumor in a cancer patient, the method comprising, or consisting essentially of, or yet further consisting of, administering to the subject any one of the cancer- or tumor-directed engineered immune cells as disclosed herein.

Certain embodiments of the engineered immune cells as disclosed herein can be useful for the treating cancer in a subject in need thereof, the method comprising, or consisting essentially of, or yet further consisting of administering to the subject any one of the cancer- or tumor-directed engineered immune cells disclosed herein.

Also provided is a method of providing anti-tumor immunity in a subject in need thereof, the method comprising, or consisting essentially of, or yet further consisting of, administering to the subject a cancer-directed engineered immune cell as disclosed herein.

In another embodiment, provided herein is a method of treating a subject in need thereof such as suffering from a disease, disorder or condition associated with the expression of or an elevated expression of a tumor antigen, the method comprising, or consisting essentially of, or yet further consisting of, administering to the subject an antigen-directed engineered immune cell as disclosed herein. Non-liming examples of the antigens of the antigen directed engineered cells are for example, the tumor antigens CD19, mesothelin, ROR1, or EGFRvIII.

Engineered immune cells directed to a cancer or tumor can be administered for decreasing, reducing, inhibiting, suppressing, limiting or controlling an adverse symptom of a neoplasia, neoplastic disorder, tumor, cancer, malignancy, metastasis of a neoplasia, tumor, cancer or malignancy to other sites, or formation or establishment of a metastatic neoplasia, neoplastic disorder, tumor, cancer or malignancy to other sites distal from a primary neoplasia, neoplastic disorder, tumor, cancer or malignancy. In some embodiments, the neoplasia, neoplastic disorder, tumor, cancer or malignancy treated is a carcinoma, sarcoma, neuroblastoma, cervical cancer, hepatocellular cancer, mesothelioma, glioblastoma, myeloma, lymphoma, leukemia, adenoma, adenocarcinoma, glioma, retinoblastoma, astrocytoma, oligodendrocytoma, meningioma, lymphosarcoma, liposarcoma, osteosarcoma, chondrosarcoma, leiomyosarcoma, rhabdomyosarcoma, fibrosarcoma or melanoma; or a cancer or neoplasia of the lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin neoplasia, neoplastic disorder, tumor, cancer or malignancy. In some embodiments, the cancer is pancreatic ductal carcinoma.

In some embodiments, an agent or treatment for cancer is administered prior to, contemporaneous with, or after treatment or diagnosis of the cancer. In certain embodiments, the administration is local or systemic. In other embodiments, the administration comprises intravenous administration. The cells and therapies as disclosed herein can be combined with other cancer therapies or treatments such as surgical resection.

In yet another aspect, a method of providing immunity to a pathogen infection in a subject in need thereof is provided, the method comprising, or consisting essentially of, or yet further consisting of, administering to the subject any one of the engineered immune cells directed to treatment of the pathogen as disclosed herein.

In further embodiments of the presently described methods, the subject is a mammal, and may be for example a mouse or a human.

### BRIEF DESCRIPTION OF THE FIGURE

FIGS. 1A to 1H shows the therapeutic use of the disclosed engineered immune cells and methods. FIG. FIG. 1A provides a non-limiting example of RelB depletion in CD8 CAR-T cells performed with a single guide RNA (sgRNA). FIG. 1B shows an example of the survival of tumor-bearing mice administered various adoptive cell therapies, including Regnase-1 and RelB depleted CAR-T cell populations. FIGS. 1C and 1D show the tumor volume overtime of those mice described in FIG. 1B. FIG. 1E shows an example of the effect of RelB depletion on CAR-T expansion and proliferation via the measurement of the CAR tumor infiltrating lymphocyte (TIL) frequency, absolute number, and density. FIG. 1F provides a non-limiting example of the expression of Ki-67 in RelB depleted CAR-T cells. FIG. 1G provides a non-limiting example of Rel-B depletion in CAR-T cells and the resulting improved production of the cytokine TNF alpha upon restimulation. FIG. 1h provides a non limiting example of increased expression of Granzyme B in Rel-B depleted CAR-T cells under resting conditions.

### DETAILED DESCRIPTION

Thus, in particular embodiments, the elements of the present disclosure may elicit, stimulate, induce, promote, increase or enhance an anti-cancer response in a subject.

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

The elements of the present disclosure can be employed in various methods, uses and compositions. Such methods and uses include, for example, use, contact or administration of one or more elements of the present disclosure in vitro and in vivo. Such methods are applicable to providing treatment to a subject for cancer or infection, immune disorder, or autoimmune response, disorder or disease.

Methods and compositions of the disclosure include administration of the diagnostics, treatments, and agents disclosed herein, to a subject alone or in combination with any compound, agent, drug, treatment or other therapeutic regimen or protocol having a desired therapeutic, beneficial, additive, synergistic or complementary activity or effect.

The disclosure therefore provides treatments in combination with a second active, including but not limited to any compound, agent, drug, therapeutic regimen, treatment protocol, process, remedy or composition, such as a treatment protocol set forth herein or known in the art. The compound, agent, drug, therapeutic regimen, treatment protocol, process, remedy or composition can be administered or performed prior to, substantially contemporaneously with or following administration of elements disclosed herein to a subject. Specific non-limiting examples of combination embodiments therefore include the foregoing or other compound, agent, drug, therapeutic regimen, treatment protocol, process, remedy or composition.

In methods of the present invention, compositions are used for which there is a desired outcome, such as a therapeutic or prophylactic method that provides a benefit from treatment, vaccination or immunization, and can be administered in a sufficient or effective amount.

### Definitions

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0, 0.7, 0.5, 0.3, 0.1, or 0.01, as appropriate, or alternatively by a variation of +/−15%, or alternatively 10%, or alternatively 5%, or alternatively 2%. It is to be understood, although not always explicitly stated, that all numerical designations are preceded by the term “about” and the appropriate range is included within the use of the term. The term “about,” as used herein when referring to a measurable value such as an amount or concentration and the like, is meant to encompass variations of 20%, 15%, 10%, 7%, 5%, 3%, 1%, 0.5%, 0.1% or even 0.01% of the specified amount. It also is to be understood, although not always explicitly stated, that the reagents described herein are merely exemplary and that equivalents of such are known in the art.

As used in the description of the invention and the appended claims, the singular forms “a,” “an” and “the” are intended to include the plural forms as well, unless the context clearly indicates otherwise.

The term “subject” refers includes but is not limited to a subject at risk of cancer or an infection, immune disorder, or autoimmune response, disorder or disease, as well as a subject that has already developed cancer or an age-associated genome dysfunction, immune disorder, or autoimmune response, disorder or disease. Such subjects, include mammalian animals (mammals), such as a non-human primate (apes, gibbons, gorillas, chimpanzees, orangutans, macaques), a domestic animal (dogs and cats), a farm animal (poultry such as chickens and ducks, horses, cows, goats, sheep, pigs), experimental animal (mouse, rat, rabbit, guinea pig) and humans. Subjects include animal disease models, for example, mouse and other animal models of cancer or an age-associated genome dysfunction, immune disorder, or autoimmune response, disorder or disease known in the art.

Accordingly, subjects appropriate for treatment include those having or at risk of cancer or an infection, immune disorder, or autoimmune response, disorder or disease, also referred to as subjects in need of treatment. Subjects in need of treatment therefore include subjects that have been previously had cancer or an infection, immune disorder, or autoimmune response, disorder or disease or that have an ongoing cancer or an infection, immune disorder, or autoimmune response, disorder or disease or have developed one or more adverse symptoms caused by or associated with cancer or an infection, immune disorder, or autoimmune response, disorder or disease, regardless of the type, timing or degree of onset, progression, severity, frequency, duration of the symptoms.

Prophylactic uses and methods are therefore included. Target subjects for prophylaxis may be at increased risk (probability or susceptibility) of developing cancer or an infection, immune disorder, or autoimmune response, disorder or disease. Such subjects are considered in need of treatment due to being at risk.

Subjects for prophylaxis need not be at increased risk but may be from the general population in which it is desired to protect a subject against cancer or an infection, immune disorder, or autoimmune response, disorder or disease, for example. Such a subject that is desired to be protected against cancer or an infection, immune disorder, or autoimmune response, disorder or disease can be administered treatment or agent described herein. In another non-limiting example, a subject that is not specifically at risk for cancer or an infection, immune disorder, or autoimmune response, disorder or disease, but nevertheless desires protection against cancer or an infection, immune disorder, or autoimmune response, disorder or disease, can be administered a composition or agent as described herein. Such subjects are also considered in need of treatment.

“Prophylaxis” and grammatical variations thereof mean a method in which contact, administration or in vivo delivery to a subject is prior to development of cancer or an infection, immune disorder, or autoimmune response, disorder or disease. In certain situations it may not be known that a subject has developed cancer or an infection, immune disorder, or autoimmune response, disorder or disease, but administration or in vivo delivery to a subject can be performed prior to manifestation of disease pathology or an associated adverse symptom, condition, complication, etc. caused by or associated with cancer or an infection, immune disorder, or autoimmune response, disorder or disease. In such case, a composition or method of the present disclosure can eliminate, prevent, inhibit, suppress, limit, decrease or reduce the probability of or susceptibility to cancer or an infection, immune disorder, or autoimmune response, disorder or disease, or an adverse symptom, condition or complication associated with or caused by cancer or an infection, immune disorder, or autoimmune response, disorder or disease.

“Prophylaxis” can also refer to a method in which contact, administration or in vivo delivery to a subject is prior to a secondary or subsequent exposure or infection. In such a situation, a subject may have had a prior cancer or an infection, immune disorder, or autoimmune response, disorder or disease or prior adverse symptom, condition or complication associated with or caused by cancer or an infection, immune disorder, or autoimmune response, disorder or disease. Treatment by administration or in vivo delivery to such a subject, can be performed prior to a secondary or subsequent cancer or an infection, immune disorder, or autoimmune response, disorder or disease. Such a method can eliminate, prevent, inhibit, suppress, limit, decrease or reduce the probability of or susceptibility towards a secondary or subsequent cancer or an infection, immune disorder, or autoimmune response, disorder or disease, or an adverse symptom, condition or complication associated with or caused by or associated with a secondary or subsequent cancer or an infection, immune disorder, or autoimmune response, disorder or disease.

Treatment of cancer or an infection, immune disorder, or autoimmune response, disorder or disease can be at any time during the cancer or an infection, immune disorder, or autoimmune response, disorder or disease. Certain embodiments of the present disclosure can be administered as a combination (e.g., with a second active), or separately concurrently or in sequence (sequentially) in accordance with the methods described herein as a single or multiple dose e.g., one or more times hourly, daily, weekly, monthly or annually or between about 1 to 10 weeks, or for as long as appropriate, for example, to achieve a reduction in the onset, progression, severity, frequency, duration of one or more symptoms or complications associated with or caused by cancer or an infection, immune disorder, or autoimmune response, disorder or disease, or an adverse symptom, condition or complication associated with or caused by cancer or an infection, immune disorder, or autoimmune response, disorder or disease. Thus, a method can be practiced one or more times (e.g., 1-10, 1-5 or 1-3 times) an hour, day, week, month, or year. The skilled artisan will know when it is appropriate to delay or discontinue administration. A non-limiting dosage schedule is 1-7 times per week, for 1, 2, 3, 4, 5, 6, 7, 8, 9, 10, 15, 20 or more weeks, and any numerical value or range or value within such ranges.

Methods of the disclosure can be practiced by any mode of administration or delivery, or by any route, systemic, regional and local administration or delivery. Exemplary administration and delivery routes include intravenous (i.v.), intraperitoneal (i.p.), intrarterial, intramuscular, parenteral, subcutaneous, intra-pleural, topical, dermal, intradermal, transdermal, transmucosal, intra-cranial, intra-spinal, rectal, oral (alimentary), mucosal, inhalation, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, or intralymphatic.

Doses can be based upon current existing protocols, empirically determined, using animal disease models or optionally in human clinical trials. Initial study doses can be based upon animal studies, e.g. a mouse, and the amount treatment or agent disclosed herein administered in an amount that is determined to be effective. Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. The dose can be adjusted according to the mass of a subject, and will generally be in a range from about 1-10 ug/kg, 10-25 ug/kg, 25-50 ug/kg, 50-100 ug/kg, 100-500 ug/kg, 500-1,000 ug/kg, 1-5 mg/kg, 5-10 mg/kg, 10-20 mg/kg, 20-50 mg/kg, 50-100 mg/kg, 100-250 mg/kg, 250-500 mg/kg, or more, two, three, four, or more times per hour, day, week, month or annually. A typical range will be from about 0.3 mg/kg to about 50 mg/kg, 0-25 mg/kg, or 1.0-10 mg/kg, or any numerical value or range or value within such ranges.

Doses can vary and depend upon whether the treatment is prophylactic or therapeutic, whether a subject has previously had cancer or an infection, immune disorder, or autoimmune response, disorder or disease, the onset, progression, severity, frequency, duration probability of or susceptibility of the symptom, condition, pathology or complication, the treatment protocol and compositions, the clinical endpoint desired, the occurrence of previous or simultaneous treatments, the general health, age, gender, race or immunological competency of the subject and other factors that will be appreciated by the skilled artisan. The skilled artisan will appreciate the factors that may influence the dosage and timing required to provide an amount sufficient for providing a therapeutic or prophylactic benefit.

The dose amount, number, frequency or duration may be proportionally increased or reduced, as indicated by the status of the subject. For example, whether the subject has previously had cancer or an infection, immune disorder, or autoimmune response, disorder or disease, whether the subject is merely at risk of cancer or an infection, immune disorder, or autoimmune response, disorder or disease, exposure or infection, whether the subject has been previously treated for cancer or an infection, immune disorder, or autoimmune response, disorder or disease. The dose amount, number, frequency or duration may be proportionally increased or reduced, as indicated by any adverse side effects, complications or other risk factors of the treatment or therapy.

In the methods of the invention, the route, dose, number and frequency of administrations, treatments, and timing/intervals between treatment and disease development can be modified. In certain embodiments, a desirable treatment of the present disclosure will elicit robust, long-lasting immunity against cancer or an infection, immune disorder, or autoimmune response, disorder or disease. Thus, in certain embodiments, disclosure methods, uses and compositions provide long-lasting immunity to cancer or an infection, immune disorder, or autoimmune response, disorder or disease.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, ophthalmic, optical, intravenous (i.v.), intramuscular, intraglandular, intraorgan, or intralymphatic.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

To increase a treatment as described herein comprising a vaccination, a composition of the present disclosure can be coupled to one or more proteins such as ovalbumin or keyhole limpet hemocyanin (KLH), thyroglobulin or a toxin such as tetanus or cholera toxin. Disclosure compositions can also be mixed with adjuvants. As demonstrated herein, in certain embodiments, the form of adjuvant with which the disclosure proteins or peptides are mixed may change whether the protein or peptide elicits an atherogenic or protective response in a subject.

Adjuvants include, for example: oil (mineral or organic) emulsion adjuvants such as Freund's complete (CFA) and incomplete adjuvant (IFA) (WO 95/17210; WO 98/56414; WO 99/12565; WO 99/11241; and U.S. Pat. No. 5,422,109); metal and metallic salts, such as aluminum and aluminum salts, such as aluminum phosphate or aluminum hydroxide, alum (hydrated potassium aluminum sulfate); bacterially derived compounds, such as Monophosphoryl lipid A and derivatives thereof (e.g., 3 De-O-acylated monophosphoryl lipid A, aka 3D-MPL or d3-MPL, to indicate that position 3 of the reducing end glucosamine is de-O-acylated, 3D-MPL consisting of the tri and tetra acyl congeners), and enterobacterial lipopolysaccharides (LPS); plant derived saponins and derivatives thereof, for example Quil A (isolated from the Quilaja Saponaria Molina tree, see, e.g., “Saponin adjuvants”, Archiv. fur die gesamte Virusforschung, Vol. 44, Springer Verlag, Berlin, p 243-254; U.S. Pat. No. 5,057,540), and fragments of Quil A which retain adjuvant activity without associated toxicity, for example QS7 and QS21 (also known as QA7 and QA21), as described in WO96/33739, for example; surfactants such as, soya lecithin and oleic acid; sorbitan esters such as sorbitan trioleate; and polyvinylpyrrolidone; oligonucleotides such as CpG (WO 96/02555, and WO 98/16247), polyriboA and polyriboU; block copolymers; and immunostimulatory cytokines such as GM-CSF and IL-1, and Muramyl tripeptide (MTP). Additional examples of adjuvants are described, for example, in “Vaccine Design—the subunit and adjuvant approach” (Edited by Powell, M. F. and Newman, M. J.; 1995, Pharmaceutical Biotechnology (Plenum Press, New York and London, ISBN 0-306-44867-X) entitled “Compendium of vaccine adjuvants and excipients” by Powell, M. F. and Newman M.

Salts may be added to a composition of the present invention. Non-limiting examples of salts include acetate, benzoate, besylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulphate, mucate, napsylate, nitrate, pamoate (embonate, phosphate, diphosphate, salicylate and disalicylate, stearate, succinate, sulphate, tartrate, tosylate, triethiodide, valerate, aluminium, benzathine, calcium, ethylene diamine, lysine, magnesium, meglumine, potassium, procaine, sodium, tromethyamine or zinc.

Chelating agents may be added to a composition of the present invention. Non-limiting examples of chelating agents include ethylenediamine, ethylene glycol tetraacetic acid, 1,2-bis(o-aminophenoxy) ethane-N,N,N′,N′-tetraacetic acid, Penicillamine, Deferasirox, Deferiprone, Deferoxamine, 2,3-Disulfanylpropan-1-ol, Dexrazoxane, Iron(II,III) hexacyanoferrate(II,III), (R)-5-(1,2-dithiolan-3-yl) pentanoic acid, 2,3-Dimercapto-1-propanesulfonic acid, Dimercaptosuccinic acid, or diethylene triamine pentaacetic acid.

Buffering agents may be added to a composition of the present invention. Non-limiting examples of buffering agents include phosphate, citrate, acetate, borate, TAPS, bicine, tris, tricine, TAPSO, HEPES, TES, MOPS, PIPES, cacodylate, SSC, MES or succinic acid.

Cosolvents may be added to a composition of the present invention. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, ribavirin, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Pharmaceutical formulations and delivery systems appropriate for the compositions and methods of the disclosure are known in the art (see, e.g., Remington: The Science and Practice of Pharmacy (2003) 20^{th} ed., Mack Publishing Co., Easton, PA; Remington's Pharmaceutical Sciences (1990) 18^{th} ed., Mack Publishing Co., Easton, PA; The Merck Index (1996) 12^{th} ed., Merck Publishing Group, Whitehouse, NJ; Pharmaceutical Principles of Solid Dosage Forms (1993), Technonic Publishing Co., Inc., Lancaster, Pa.; Ansel ad Soklosa, Pharmaceutical Calculations (2001) 11^{th} ed., Lippincott Williams & Wilkins, Baltimore, MD; and Poznansky et al., Drug Delivery Systems (1980), R. L. Juliano, ed., Oxford, N.Y., pp. 253-315).

An agent as described herein can be packaged in unit dosage form (capsules, tablets, troches, cachets, lozenges) for ease of administration and uniformity of dosage. A “unit dosage form” as used herein refers to physically discrete units suited as unitary dosages for the subject to be treated; each unit containing a predetermined quantity of active ingredient optionally in association with a pharmaceutical carrier (excipient, diluent, vehicle or filling agent) which, when administered in one or more doses, is calculated to produce a desired effect (e.g., prophylactic or therapeutic effect). Unit dosage forms also include, for example, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Individual unit dosage forms can be included in multi-dose kits or containers. Pharmaceutical formulations can be packaged in single or multiple unit dosage form for ease of administration and uniformity of dosage.

As used herein, numerical values are often presented in a range format throughout this document. The use of a range format is merely for convenience and brevity and should not be construed as an inflexible limitation on the scope of the invention. Accordingly, the use of a range expressly includes all possible subranges, all individual numerical values within that range, and all numerical values or numerical ranges include integers within such ranges and fractions of the values or the integers within ranges unless the context clearly indicates otherwise. This construction applies regardless of the breadth of the range and in all contexts throughout this patent document. Thus, to illustrate, reference to a range of 90-100% includes 91-99%, 92-98%, 93-95%, 91-98%, 91-97%, 91-96%, 91-95%, 91-94%, 91-93%, and so forth. Reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5 fold therefore includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth. Further, for example, reference to a series of ranges of 2-72 hours, 2-48 hours, 4-24 hours, 4-18 hours and 6-12 hours, includes ranges of 2-6 hours, 2, 12 hours, 2-18 hours, 2-24 hours, etc., and 4-27 hours, 4-48 hours, 4-6 hours, etc.

As also used herein a series of range formats are used throughout this document. The use of a series of ranges includes combinations of the upper and lower ranges to provide a range. Accordingly, a series of ranges include ranges which combine the values of the boundaries of different ranges within the series. This construction applies regardless of the breadth of the range and in all contexts throughout this patent document. Thus, for example, reference to a series of ranges such as 5-10, 10-20, 20-30, 30-40, 40-50, 50-75, 75-100, 100-150, and 150-171, includes ranges such as 5-20, 5-30, 5-40, 5-50, 5-75, 5-100, 5-150, 5-171, and 10-30, 10-40, 10-50, 10-75, 10-100, 10-150, 10-171, and 20-40, 20-50, 20-75, 20-100, 20-150, 20-171, and so forth.

The term “isolated” as used herein with respect to nucleic acids, such as DNA or RNA, refers to molecules separated from other DNAs or RNAs, respectively that are present in the natural source of the macromolecule. The term “isolated nucleic acid” is meant to include nucleic acid fragments which are not naturally occurring as fragments and would not be found in the natural state. The term “isolated” is also used herein to refer to polypeptides, proteins and/or host cells that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

In some embodiments, the term “engineered” or “recombinant” refers to having at least one modification not normally found in a naturally occurring protein, polypeptide, polynucleotide, strain, wild-type strain or the parental host strain of the referenced species. In some embodiments, the term “engineered” or “recombinant” refers to being synthetized by human intervention. As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

The terms “polynucleotide”, “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The expression “amplification of polynucleotides” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or its flanking regions. Nucleic acid sequences generated by amplification may be sequenced directly. Alternatively, the amplified sequence(s) may be cloned prior to sequence analysis. A method for the direct cloning and sequence analysis of enzymatically amplified genomic segments is known in the art.

A “gene” refers to a polynucleotide containing at least one open reading frame (ORF) that is capable of encoding a particular polypeptide or protein after being transcribed and translated.

The term “express” refers to the production of a gene product, such as mRNA, peptides, polypeptides or proteins. As used herein, “expression” refers to the process by which polynucleotides are transcribed into mRNA or the process by which the transcribed mRNA is subsequently being translated into peptides, polypeptides, or proteins. If the polynucleotide is derived from genomic DNA, expression may include splicing of the mRNA in a eukaryotic cell.

As used herein, the term “overexpress” intends a level of expression of the mRNA, the protein or the polypeptide” that is greater than or exceeds the level of expression of the mRNA, the protein or the polypeptide in a native, wild-type or cell that has not been engineered to increase expression.

A “gene product” or alternatively a “gene expression product” refers to the amino acid (e.g., peptide or polypeptide) generated when a gene is transcribed and translated. In some embodiments, the gene product may refer to an mRNA or other RNA, such as an interfering RNA, generated when a gene is transcribed.

The term “encode” as it is applied to polynucleotides refers to a polynucleotide which is said to “encode” a polypeptide if, in its native state or when manipulated by methods well known to those skilled in the art, it can be transcribed to produce the mRNA for the polypeptide or a fragment thereof, and optionally translated to produce the polypeptide or a fragment thereof. The antisense strand is the complement of such a nucleic acid, and the encoding sequence can be deduced therefrom. Further, as used herein an amino acid sequence coding sequence refers to a nucleotide sequence encoding the amino acid sequence.

“Under transcriptional control”, which is also used herein as “directing expression of”, is a term well understood in the art and indicates that transcription of a polynucleotide sequence, usually a DNA sequence, depends on its being operatively linked to an element which contributes to the initiation of, or promotes, transcription. “Operatively linked” intends the polynucleotides are arranged in a manner that allows them to function in a cell.

The term “a regulatory sequence”, “an expression control element” or “promoter” as used herein, intends a polynucleotide that is operatively linked to a target polynucleotide to be transcribed or replicated, and facilitates the expression or replication of the target polynucleotide. A promoter is an example of an expression control element or a regulatory sequence. Promoters can be located 5′ or upstream of a gene or other polynucleotide, that provides a control point for regulated gene transcription. Polymerase II and III are examples of promoters. In some embodiments, a regulatory sequence is bidirectional, i.e., acting as a regulatory sequence for the coding sequences on both sides of the regulatory sequence. Such bidirectional regulatory sequence may comprise, or consists essentially of, or consists of a bidirectional promoter (see for example Trinklein N D, et al. (2004) An abundance of bidirectional promoters in the human genome. Genome Res. January; 14(1):62-6).

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting examples of promoters include the EF1alpha promoter and the CMV promoter. The EF1alpha sequence is known in the art (see, e.g., addgene.org/11154/sequences/; ncbi.nlm.nih.gov/nuccore/J04617, each last accessed on Mar. 13, 2019, and Zheng and Baum (2014) Int'l. J. Med. Sci. 11(5):404-408). The CMV promoter sequence is known in the art (see, e.g., snapgene.com/resources/plasmid-files/?set=basic_cloning_vectors&plasmid=CMV_promoter, last accessed on Mar. 13, 2019 and Zheng and Baum (2014), supra.).

The term “protein”, “peptide” and “polypeptide” are used interchangeably and in their broadest sense to refer to a compound of two or more subunit amino acids, amino acid analogs or peptidomimetics. The subunits (which are also referred to as residues) may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

RELB (RELB Proto-Oncogene, NF-KB Subunit) is a protein coding gene. Diseases associated with RELB function or dysfunction include Immunodeficiency 53 and Rheumatoid Arthritis. Among its related pathways are immune response NFAT in immune response and IL-1 Family Signaling Pathways. Gene Ontology (GO) annotations related to this gene include DNA-binding transcription factor activity and transcription corepressor activity. An important paralog of this gene is RELA. NF-kappa-B is a pleiotropic transcription factor which is present in almost all cell types and is involved in many biological processed such as inflammation, immunity, differentiation, cell growth, tumorigenesis and apoptosis. NF-kappa-B is a homo- or heterodimeric complex formed by the Rel-like domain-containing proteins RELA/p65, RELB, NFKB1/p105, NFKB1/p50, REL and NFKB2/p52. The dimers bind at kappa-B sites in the DNA of their target genes and the individual dimers have distinct preferences for different kappa-B sites that they can bind with distinguishable affinity and specificity. Different dimer combinations act as transcriptional activators or repressors, respectively. NF-kappa-B is controlled by various mechanisms of post-translational modification and subcellular compartmentalization as well as by interactions with other cofactors or corepressors. NF-kappa-B complexes are held in the cytoplasm in an inactive state complexed with members of the NF-kappa-B inhibitor (I-kappa-B) family. In a conventional activation pathway, I-kappa-B is phosphorylated by I-kappa-B kinases (IKKs) in response to different activators, subsequently degraded thus liberating the active NF-kappa-B complex which translocates to the nucleus. NF-kappa-B heterodimeric RelB-p50 and RelB-p52 complexes are transcriptional activators. RELB neither associates with DNA nor with RELA/p65 or REL. Stimulates promoter activity in the presence of NFKB2/p49. As a member of the NUPR1/RELB/IER3 survival pathway, may provide pancreatic ductal adenocarcinoma with remarkable resistance to cell stress, such as starvation or gemcitabine treatment. Regulates the circadian clock by repressing the transcriptional activator activity of the CLOCK-ARNTL/BMAL1 heterodimer in a CRY1/CRY2 independent manner. Increased repression of the heterodimer is seen in the presence of NFKB2/p52. Is required for both T and B lymphocyte maturation and function.

A non-limiting example of RelB human polypeptide amino acid sequence is disclosed in UniProtKB/Swiss-Prot Ref. Sequence: Q01201reproduced below (SEQ ID NO: 1):

MLRSGPASGPSVPTGRAMPSRRVARPPAAPELGALGSPDLSSLSL
AVSRSTDELEIIDEYIKENGFGLDGGQPGPGEGLPRLVSRGAASL
STVTLGPVAPPATPPPWGCPLGRLVSPAPGPGPQPHLVITEQPKQ
RGMRFRYECEGRSAGSILGESSTEASKTLPAIELRDCGGLREVEV
TACLVWKDWPHRVHPHSLVGKDCTDGICRVRLRPHVSPRHSFNNL
GIQCVRKKEIEAAIERKIQLGIDPYNAGSLKNHQEVDMNVVRICF
QASYRDQQGQMRRMDPVLSEPVYDKKSTNTSELRICRINKESGPC
TGGEELYLLCDKVQKEDISVVFSRASWEGRADFSQADVHRQIAIV
FKTPPYEDLEIVEPVTVNVFLQRLTDGVCSEPLPFTYLPRDHDSY
GVDKKRKRGMPDVLGELNSSDPHGIESKRRKKKPAILDHFLPNHG
SGPFLPPSALLPDPDFFSGTVSLPGLEPPGGPDLLDDGFAYDPTA
PTLFTMLDLLPPAPPHASAVVCSGGAGAVVGETPGPEPLTLDSYQ
APGPGDGGTASLVGSNMFPNHYREAAFGGGLLSPGPEAT

Isoforms of RelB are also disclosed herein, and include:

(SEQ ID NO: 32):
MLRSGPASGPSVPTGRAMPSRRVARPPAAPELGALGSPDLSSLSL
AVSRSTGSSSTDA.
Disclosed in UniProtKB/Swiss-Prot
Ref. Sequence: D6RIV7.
(SEQ ID NO: 33)
MLRSGPASGPSVPTGRAMPSRRVARPPAAPELGALGSPDLSSLSL
AVSRSTEIIDEYIKENGFGLDGGQPGPGEGLPRLVSRGAASLSTV
TLGPVAPPATPPPWGCPLGRLVSPAPGPGPQPHLVITEQPKQRGM
RFRYECEGRSAGSILGESSTEASKTLPAIELRDCGGLREVEVTAC
LVWKDWPHRVHPHSLVGKDCTDGICRVRLRPHVSPRHSFNNLGIQ
CVRKKEIEAAIERKIQLGIDPYNAGSLKNHQEVDMNVVRICFQAS
YRDQQGQMRRMDPVLSEPVYDKKSTNTSELRICRINKESGPCTGG
EELYLLCDKVQKEDISVVFSRASWEGRADFSQADVHRQIAIVFKT
PPYEDLEIVEPVTVNVFLQRLTDGVCSEPLPFTYLPRDHDSYGVD
KKRKRGMPDVLGELNSSDPHGIESKRRKKKPAILDHFLPNHGSGP
FLPPSALLPDPDFFSGTVSLPGLEPPGGPDLLDDGFAYDPTAPTL
FTMLDLLPPAPPHASAVVCSGGAGAVVGETPGPEPLTLDSYQAPG
PGDGGTASLVGSNMFPNHYREAAFGGGLLSPGPEAT.
Disclosed in UniProtKB/Swiss-Prot Ref.
Sequence: D6RIV7.
(SEQ ID NO: 34):
EIIDEYIKENGFGLDGGQPGPGEGLPRLVSRGAASLSTVTLGPVA
PPATPPPWGCPLGRLVSPAPGPGPQPHLVITEQPKQRGMRFRYEC
EGRSAGSILGESSTEASKTLPAIEF.
Disclosed in UniProtKB/Swiss-Prot Ref.
Sequence: A0A8V8TQY2.
(SEQ ID NO: 35):
XSEPLPFTYLPRDHGPFLPPSALLPDPDFFSGTVSLPGLEPPGGP
DLLDDGFAYDPTAPTLFTMLDLLPPAPPHASAVVCSGGAGAVVGE
TPGPEPLTLDSYQAPGPGDGGTASLVGSNMFPNHYREAAFGGGLL
SPGPEAT.
Disclosed in UniProtKB/Swiss-Prot Ref.
Sequence: K7ERX9

Thymocyte Selection Associated High Mobility Group Box (TOX) is a transcriptional regulator that plays a role in neural stem cell commitment and lymphoid cell development. TOX binds to GC-rich DNA sequences in the proximity of transcription start sites and may alter chromatin structure, modifying access of transcription factors to DNA. TOX may be required for the development of various T cell subsets, including CD4-positive helper T cells, CD8-positive cytotoxic T cells, regulatory T cells and CD1D-dependent natural killer T (NKT) cells and may be required at the progenitor phase of NK cell development in the bone marrow to specify NK cell lineage commitment. Upon chronic antigen stimulation, TOX diverts T cell development by promoting the generation of exhaustive T cells, while suppressing effector and memory T cell programming.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample). The term “antibody” also includes genetically engineered forms such as chimeric antibodies (for example, murine or humanized non-primate antibodies), heteroconjugate antibodies (such as, bispecific antibodies). See also, Pierce Catalog and Handbook, 1994-1995 (Pierce Chemical Co., Rockford, Ill.); Owen et al., Kuby Immunology, 7th Ed., W.H. Freeman & Co., 2013; Murphy, Janeway's Immunobiology, 8th Ed., Garland Science, 2014; Male et al., Immunology (Roitt), 8th Ed., Saunders, 2012; Parham, The Immune System, 4th Ed., Garland Science, 2014. In some embodiments, the term “antibody” refers to a single-chain variable fragment (scFv or ScFV). In some embodiments, the term “antibody” refers to more than one single-chain variable fragments (scFv, or ScFV) linked with each other, optionally via a peptide linker or another suitable component as disclosed herein. In some embodiments, an antibody is a monoclonal antibody. In some embodiments, an antibody is a monospecific antibody or a multispecific antibody, such as a bispecific antibody or a trispecific antibody. The species of the antibody can be a human or non-human, e.g., mammalian

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located (heavy chain regions labeled CDRH and light chain regions labeled CDRL). Thus, a CDRH3 is the CDR3 from the variable domain of the heavy chain of the antibody in which it is found, whereas a CDRL1 is the CDR1 from the variable domain of the light chain of the antibody in which it is found. For example, an anti-CD19 antibody will have a specific VH region and the VL region sequence unique to the CD19 relevant antigen, and thus specific CDR sequences. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, a single-chain variable fragment (scFv or ScFV), also referred to herein as a fragment of an antibody, and is a fusion protein of the variable regions of the heavy (V_{H}) and light chains (V_{L}) of immunoglobulins, optionally connected with a short linker peptide of about 10 to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker.

As used herein, a fragment crystallizable (Fc) region refers to the tail region of an antibody that stabilizes the antibody, and optionally interacts with (such as binds) an Fc receptor on an immune cell or on a platelet or that binds a complement protein. In some embodiments, a Fc mutant may be used, such as comprising one or two or all three mutations of F234A, L235A and N297Q of human IgG4 Fc region in a Fc or an equivalent thereof at positions corresponding to those of human IgG4 Fc region, such as for ESKYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLPS SIEKTISKAKGQPREPQVYTLPPSQEEMTKNQVSLTCLVKGFYPSDIAVEWESNGQP ENNYKTTPPVLDSDGSFFLYSRLTVDKSRWQEGNVFSCSVMHEALHNHYTQKSLS LSLGK (SEQ ID NO: 2), the corresponding positions are amino acid (aa) 16, aa 17 and aa 79 of ESKYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLPS SIEKTISKAKGQPREPQVYTLPPSQEEMTKNQVSLTCLVKGFYPSDIAVEWESNGQP ENNYKTTPPVLDSDGSFFLYSRLTVDKSRWQEGNVFSCSVMHEALHNHYTQKSLS LSLGK (SEQ ID NO: 3). As shown in Wang et al. Protein Cell. 2018 January; 9(1):63-73. Epub 2017 Oct. 6 and other publications, one of skill in the art would engineers an Fc region according to the use, such as reducing inflammatory cytokine release etc.

The polypeptide or an equivalent thereof, can be followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus (C-terminus). Additionally or alternatively, the polypeptide or an equivalent thereof can further comprises an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the amine-terminus (N-terminus).

An equivalent of a reference polypeptide comprises, consists essentially of, or alternatively consists of an polypeptide having at least 80% amino acid identity to the reference polypeptide, such as the CAR as disclosed herein, or a polypeptide that is encoded by a polynucleotide that hybridizes under conditions of high stringency to the complement of a polynucleotide encoding the reference polypeptide, such as a CAR as disclosed herein, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

By “fragment” is intended a molecule consisting of only a part of the intact full-length sequence and structure. The fragment of a polypeptide can include a C-terminal deletion, an N-terminal deletion, an internal deletion of the native polypeptide, or any combination thereof. Active fragments of a particular protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence, provided that the fragment in question substantially retains biological activity.

Alternative embodiments include one or more of the CDRs (e.g., CDR1, CDR2, CDR3) from the LC variable region substituted with appropriate CDRs from other antibody CDRs, or an equivalent of each thereof. Accordingly, and as an example, the CDR1 and CDR2 from the LC variable region can be combined with the CDR3 of another antibody's LC variable region, and in some aspects, can include an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the term “equivalent” or “biological equivalent” of an antibody means the ability of the antibody to selectively bind its epitope protein or a fragment thereof as measured by ELISA or other suitable methods is substantively maintained, for example, at a level of at least 50%, or at least 55%, or at least 60%, or at least 65%, or at least 70%, or at least 75%, or at least 80%, or at least 85%, or at least 90%, or at least 95%, or at least 99%, or more. Biologically equivalent antibodies include, but are not limited to, those antibodies, peptides, antibody fragments, antibody variant, antibody derivative and antibody mimetics that bind to the same epitope as the reference antibody. Additionally or alternatively, the equivalent and the reference antibody shares the same set of CDRs but other amino acids are modified.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity, or at least about 85% homology or identity, or alternatively at least about 90% homology or identity, or alternatively at least about 95% homology or identity, or alternatively 98% homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

The term “antibody variant” intends to include antibodies produced in a species other than a mouse. It also includes antibodies containing post-translational modifications to the linear polypeptide sequence of the antibody or a fragment thereof. It further encompasses fully human antibodies.

The term “antibody derivative” is intended to encompass molecules that bind an epitope as defined above and which are modifications or derivatives of a native monoclonal antibody of this disclosure. Derivatives include, but are not limited to, for example, bispecific, multispecific, heterospecific, trispecific, tetraspecific, multispecific antibodies, diabodies, chimeric, recombinant and humanized.

As used herein, the term “specific binding” or “binding” means the contact between an antibody and an antigen with a binding affinity of at least 10⁻⁶ M. In certain embodiments, antibodies bind with affinities of at least about 10⁻⁷ M, and preferably at least about 10⁻⁸ M, at least about 10⁻⁹ M, at least about 10⁻¹⁰ M, at least about 10⁻¹¹ M, or at least about 10⁻¹² M.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. In some embodiments, one or more plasmids are used in producing a viral vector or a viral genome. In some embodiments, a plasmid is used for replicating or amplifying a polynucleotide. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “viral vector” is defined as a recombinantly produced virus or viral particle that comprises a polynucleotide (a viral genome) to be delivered into a host cell, either in vivo, ex vivo or in vitro or ex vivo. As is known to those of skill in the art, there are 6 classes of viruses. The DNA viruses constitute classes I and II. The RNA viruses and retroviruses make up the remaining classes. Class III viruses have a double-stranded RNA genome. Class IV viruses have a positive single-stranded RNA genome, the genome itself acting as mRNA Class V viruses have a negative single-stranded RNA genome used as a template for mRNA synthesis. Class VI viruses have a positive single-stranded RNA genome but with a DNA intermediate not only in replication but also in mRNA synthesis. Retroviruses carry their genetic information in the form of RNA; however, once the virus infects a cell, the RNA is reverse-transcribed into the DNA form which optionally integrates into the genomic DNA of the infected cell. The integrated DNA form is called a provirus.

Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In several embodiments, the vector is derived from or based on a wild-type virus. In further embodiments, the vector is derived from or based on one or more of a wild-type adenovirus, an adeno-associated virus, or a retrovirus such as a gammaretrovirus or a lentivirus. As used herein, the vector may be a gammaretroviral vector (PCIR). Examples of retrovirus include without limitation, Moloney murine leukemia virus (MMLV), murine stem cell virus (MSCV), or friend murine embryonic stem cell virus (FMEV), human immunodeficiency virus (HIV), equine infectious anaemia virus (EIAV), simian immunodeficiency virus (SIV) and feline immunodeficiency virus (FIV). The viral vector may comprise components derived from two or more different viruses, and may also comprise synthetic components. Vector components can be manipulated to obtain desired characteristics such as target cell specificity.

The recombinant vectors of this disclosure may be derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV), the more recently described feline immunodeficiency virus (FIV), and bovine immunodeficiency virus (BIV). Prior art recombinant lentiviral vectors are known in the art, e.g., see U.S. Pat. Nos. 6,924,123; 7,056,699; 7,419,829 and 7,442,551, incorporated herein by reference. In some embodiments, the lentiviral vector is a self-inactivating lentiviral vector. In further embodiments, the lentiviral vector has a U3 region lacking a TATA box. Additionally or alternatively, the lentiviral vector has a U3 region lacking one or more of transcription factor binding site(s).

A retrovirus such as a gammaretrovirus or a lentivirus comprises (a) envelope comprising lipids and glycoprotein, (b) a vector genome, which is a RNA (usually a dimer RNA comprising a cap at the 5′ end and a polyA tail at the 3′ end flanked by LTRs) delivered to the target cell, (c) a capsid, and (d) other proteins, such as a protease. U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector genome (such as an RNA vector genome) is expressed from a DNA construct (such as a plasmid) encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

In embodiments where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this disclosure are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the disclosure may be based on a genetically or otherwise (e.g. by specific choice of packaging cell system) altered version of a particular retrovirus.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11 sequentially numbered, AAV serotypes are known in the art. Non-limiting exemplary serotypes useful in the methods disclosed herein include any of the 11 serotypes, e.g., AAV2, AAV8, AAV9, or variant or synthetic serotypes, e.g., AAV-DJ and AAV PHP.B. The AAV particle comprises, alternatively consists essentially of, or yet further consists of three major viral proteins: VP1, VP2 and VP3. In one embodiment, the AAV refers to of the serotype AAV1, AAV2, AAV4, AAV5, AAV6, AAV7, AAV8, AAV9, AAV10, AAV11, AAV12, AAV13, AAV PHP.B, or AAV rh74. These vectors are commercially available or have been described in the patent or technical literature.

That the vector particle according to the disclosure is “based on” a particular retrovirus means that the vector is derived from that particular retrovirus. The genome of the vector particle comprises components from that retrovirus as a backbone. The vector particle contains essential vector components compatible with the genome, such as an RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

As used herein, “immune cells” includes, e.g., white blood cells (leukocytes, such as granulocytes (neutrophils, eosinophils, and basophils), monocytes, and lymphocytes (T cells, B cells, natural killer (NK) cells and NKT cells)) which may be derived from hematopoietic stem cells (HSC) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSc), hematopoietic stem cells (HSCs), or immortalized cells. In some embodiments, the HSC are derived from umbilical cord blood of a subject, peripheral blood of a subject, or bone marrow of a subject.

An “engineered immune cell” includes an immune cell engineered to include at least one modification not normally found in a naturally occurring protein, polypeptide, polynucleotide, strain, wild-type strain or the parental host strain of the referenced species. The engineered immune cell may be synthetized by human intervention to incorporate one or more transgenes expressing a recombinant protein (i.e.: RelB).

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are another commercially available source of immune cells, as well as cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC (www.atcc.org/) and the German Collection of Microorganisms and Cell Cultures (www.dsmz.de/).

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92 MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC (www.atcc.org/) and the German Collection of Microorganisms and Cell Cultures (www.dsmz.de/).

As used herein, the term “transmembrane domain” refers to a protein region that is hydrophobic, so that it prefers to be inserted into the cell membrane such that the parts of the protein on either side of the domain are on opposite sides of the membrane. In some embodiments, the transmembrane domain comprises, or consists essentially of, or yet further consists of a transmembrane segment of single alpha helix of a transmembrane protein. Additionally or alternatively, a transmembrane domain comprises, or consists essentially of, or yet further consists of predominantly of nonpolar amino acid residues and may traverse the membrane bilayer once or several times.

As used herein, the term “suicide gene” refers to any gene that expresses a product (optionally with presence of another agent, such as an antibody) that is fatal to the cell expressing the suicide gene. Transcription or expression of such gene, i.e., presence of its gene product, in a cell alone or together with other agents causing the cell to kill itself, for example through apoptosis. It provides a possible strategy of eliminating a cell, for example, a therapeutic cell expressing CAR, after it performs its desired function, such as treating a cancer. In further embodiments, the suicide gene product is selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR (tEGFR), or inducible caspase (“iCasp”). In yet further embodiments, exemplified suicide strategy includes the thymidine kinase/ganciclovir system, the cytosine deaminase/5-fluorocytosine system, the nitroreductase/CB1954 system, carboxypeptidase G2/Nitrogen mustard system, cytochrome P450/oxazaphosphorine system, purine nucleoside phosphorylase/6-methylpurine deoxyriboside (PNP/MEP), the horseradish peroxidase/indole-3-acetic acid system (HRP/IAA), and the carboxylesterase/irinotecan (CE/irinotecan) system, the truncated EGFR (EGFR), inducible caspase (“iCasp”), the E. coli gpt gene, the E. coli Deo gene and nitroreductase. See, more details at Karjoo, Z. et al. 2016. Adv. Drug Deliv. Rev. 99 (Pt. A):123-128.

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains. Non-limiting exemplary polynucleotide sequences that encode for components of each domain are disclosed herein, e.g.:

Hinge domain: IgG1 heavy chain hinge sequence: CTCGAGCCCAAATCTTGTGACAAAACTCACACATGCCCACCGTGCCCG (SEQ ID NO: 7), and optionally an equivalent thereof.

Transmembrane domain: CD28 transmembrane region: TTTTGGGTGCTGGTGGTGGTTGGTGGAGTCCTGGCTTGCTATAGCTTGCTAGTAA CAGTGGCCTTTATTATTTTCTGGGTG (SEQ ID NO: 8), and optionally an equivalent thereof.

Intracellular domain: 4-1BB co-stimulatory signaling region: AAACGGGGCAGAAAGAAACTCCTGTATATATTCAAACAACCATTTATGAGACC AGTACAAACTACTCAAGAGGAAGATGGCTGTAGCTGCCGATTTCCAGAAGAAG AAGAAGGAGGATGTGAACTG (SEQ ID NO: 9), and optionally an equivalent thereof.

Intracellular domain: CD28 co-stimulatory signaling region: AGGAGTAAGAGGAGCAGGCTCCTGCACAGTGACTACATGAACATGACTCCCCG CCGCCCCGGGCCCACCCGCAAGCATTACCAGCCCTATGCCCCACCACGCGACTT CGCAGCCTATCGCTCC (SEQ ID NO: 10), and optionally an equivalent thereof.

Intracellular domain: CD3 zeta signaling region: AGAGTGAAGTTCAGCAGGAGCGCAGACGCCCCCGCGTACCAGCAGGGCCAGAA CCAGCTCTATAACGAGCTCAATCTAGGACGAAGAGAGGAGTACGATGTTTTGG ACAAGAGACGTGGCCGGGACCCTGAGATGGGGGGAAAGCCGAGAAGGAAGAA CCCTCAGGAAGGCCTGTACAATGAACTGCAGAAAGATAAGATGGCGGAGGCCT ACAGTGAGATTGGGATGAAAGGCGAGCGCCGGAGGGGCAAGGGGCACGATGG CCTTTACCAGGGTCTCAGTACAGCCACCAAGGACACCTACGACGCCCTTCACAT GCAGGCCCTGCCCCCTCGCTAA (SEQ ID NO: 11), and optionally an equivalent thereof.

A chimeric antigen receptor may optionally comprise a “hinge domain” which serves as a linker between the extracellular and transmembrane domains. Non limiting examples of such domains are provided herein, e.g.:

As used herein, the term IgG1 hinge domain also refers to a specific protein fragment associated with this name or any other molecules that have analogous biological function that share at least about 70%, or alternatively at least 80% amino acid sequence identity, preferably at least about 90% sequence identity, more preferably at least about 95% sequence identity with the IgG1 hinge domain sequence as shown herein. Additional example sequences of IgG1 hinge domain are provided in, e.g., US20180273642A1 and Dall'Acqua W F, Cook K E, Damschroder M M, Woods R M, Wu H. Modulation of the effector functions of a human IgG1 through engineering of its hinge region. J Immunol. 2006 Jul. 15; 177(2):1129-38. Additional non-limiting example of a hinge domain includes those of another immunoglobulin, such as an IgG4 hinge region, and an IgD hinge domain. See, for example, US20180273642A1. Another example is a CD8 hinge domain, such as a CD8 α hinge domain, as known in the art.

Non-limiting examples of CAR extracellular domains capable of binding to antigens are the anti-CD19 binding domain sequences that specifically bind CD19 antigen as disclosed in the U.S. Application Publication No. 20140271635 and U.S. Pat. No. 7,109,304. Additional examples (e.g., anti-BCMA, mesothelin, ROR1 and EGFRvIII) are provided herein and are well known in the art.

Further embodiments of each exemplary domain component include other proteins that have analogous biological function that share at least 70%, or alternatively at least 80% amino acid sequence identity, preferably 90% sequence identity, more preferably at least 95% sequence identity with the proteins encoded by the above disclosed nucleic acid sequences. Further, non-limiting examples of such domains are provided herein.

As used herein, the term “CD8 α hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α hinge domain sequence as shown herein. The example sequences of CD8 α hinge domain for human, mouse, and other species are provided in Pinto, R. D. et al. (2006) Vet. Immunol. Immunopathol. 110:169-177. The sequences associated with the CD8 α hinge domain are provided in Pinto, R. D. et al. (2006) Vet. Immunol. Immunopathol. 110:169-177. Non-limiting examples of such include:

Human CD8 alpha hinge domain: PAKPTTTPAPRPPTPAPTIASQPLSLRPEACRPAAGGAVHTRGLDFACDIY (SEQ ID NO: 12), and optionally an equivalent thereof.

Mouse CD8 alpha hinge domain: KVNSTTTKPVLRTPSPVHPTGTSQPQRPEDCRPRGSVKGTGLDFACDIY (SEQ ID NO: 13), and optionally an equivalent thereof.

Cat CD8 alpha hinge domain: PVKPTTTPAPRPPTQAPITTSQRVSLRPGTCQPSAGSTVEASGLDLSCDIY (SEQ ID NO: 14), and optionally an equivalent thereof.

As used herein, the term “CD8 α transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α transmembrane domain sequence as shown herein. The fragment sequences associated with the amino acid positions 183 to 203 of the human T-cell surface glycoprotein CD8 alpha chain (GenBank Accession No: NP_001759.3), or the amino acid positions 197 to 217 of the mouse T-cell surface glycoprotein CD8 alpha chain (GenBank Accession No: NP_001074579.1), and the amino acid positions 190 to 210 of the rat T-cell surface glycoprotein CD8 alpha chain (GenBank Accession No: NP_113726.1) provide additional example sequences of the CD8 α transmembrane domain. The sequences associated with each of the listed accession numbers are provided as follows:

Human CD8 alpha transmembrane domain: IYIWAPLAGTCGVLLLSLVIT (SEQ ID NO: 15), and optionally an equivalent thereof.

Mouse CD8 alpha transmembrane domain: IWAPLAGICVALLLSLIITLI (SEQ ID NO: 16), and optionally an equivalent thereof.

Rat CD8 alpha transmembrane domain: IWAPLAGICAVLLLSLVITLI (SEQ ID NO: 17), and optionally an equivalent thereof.

A protein expressed on cell surface may be used as a marker (such as for purification or detection or tracking) or to provide a suicide switch of a CAR expressing cell as disclosed herein. Such protein is referred to herein as a suicide gene product or a detectable marker or both. A portion of or the whole cytoplasmic region of such protein is usually truncated so that the native function of the protein is reduced or even abolished. Thus, such a protein is also referred to herein as a truncated protein marker. In some embodiments, when used as a suicide switch of the CAR expressing cell, the truncated protein marker does not express or is expressed at a substantially lower level on a normal cell or a normal cell adjacent to the CAR expressing cell in the subject. Accordingly, upon removal of the CAR expressing cell (for example, by administering an antibody specially recognizing and binding the truncated protein marker, or by administering a toxin conjugated to a moiety directing the toxin to the truncated protein marker), a normal cell of the subject would not be jeopardized. Accordingly, in some embodiments, a method as disclosed herein can further comprise administering the subject an agent reducing or abolishing the CAR expressing cell in the subject. In further embodiments, the agent reducing or abolishing the CAR expressing cell in the subject comprises, or consists essentially of, or yet further consists of an antibody or a fragment thereof specifically recognizing and binding to the suicide gene product, such as tEGFR or RQR8. Additionally or alternatively, the administration of the agent reducing or abolishing the CAR expressing cell in the subject is about 1 day, about 3 days, about 1 week, about 2 weeks, about 3 weeks, about 4 weeks, about 5 weeks, about 6 weeks, about 7 weeks, about 8 weeks, about 9 weeks, about 10 weeks, about 3 months, about 4 months, about 5 months, about 6 months, about 1 year, about 1.5 years, about 2 years, or longer post the administration of a cell as disclosed herein. In some embodiments, antigen of a binding moiety, such as an antibody, an antigen binding fragment thereof, or a CAR, may be provided herein in a format of “antigen” followed by the binding moiety (such as a CD19 CAR), or having “anti” or “anti-” before the antigen and the binding moiety after the antigen (such as an anti-CD19 antibody), or the binding moiety followed by “to” or “directed to” and then the antigen (such as an antibody to CD19).

CD19 functions as co-receptor for the B-cell antigen receptor complex (BCR) on B-lymphocytes. It decreases the threshold for activation of downstream signaling pathways and for triggering B-cell responses to antigens and is required for normal B cell differentiation and proliferation in response to antigen challenges. See, for example, de Rie et al., Cell Immunol. 1989 February; 118(2):368-81; and Carter and Fearon. Science. 1992 Apr. 3; 256(5053):105-7. The majority of B cell malignancies, such as Non-Hodgkin's Lymphoma (NHL), acute lymphoblastic leukemia (ALL), and chronic lymphocytic leukemia (CLL), express normal to high levels of CD19. In some embodiments, the CD19 is a human CD19. Non-limiting exemplary sequences of this protein or the underlying gene can be found under Gene Cards ID: GC16P033267, HGNC: 1633, NCBI Entrez Gene: 930, Ensembl: ENSG00000177455, OMIM®: 107265, or UniProtKB/Swiss-Prot: P15391, each of which is incorporated by reference herein in its entirety.

As used herein, the term “CD28 transmembrane domain” refers to a specific protein fragment associated with this name or any other molecules that have analogous biological function that share at least about 70%, or alternatively at least about 80% amino acid sequence identity, or alternatively at least about 90% sequence identity, or alternatively at least about 95% sequence identity with the CD28 transmembrane domain sequence as shown herein. The fragment sequences associated with the GenBank Accession Nos: XM_006712862.2 or XM_009444056.1 provide additional, non-limiting, exemplified sequences of the CD28 transmembrane domain. The sequences associated with each of the listed accession numbers are provided herein, for example, transmembrane domain: CD28 transmembrane region coding sequence: TTTTGGGTGCTGGTGGTGGTTGGTGGAGTCCTGGCTTGCTATAGCTTGCTAGTAA CAGTGGCCTTTATTATTTTCTGGGTG (SEQ ID NO: 19) or a CD28 transmembrane region amino acid sequence comprising, consisting essentially of, or consisting of FWVLVVVGGVLACYSLLVTVAFIIFWV (SEQ ID NO: 20) or an equivalent thereof.

As used herein, the term “4-1BB costimulatory signaling region” or “4-1BB costimulatory region” refers to a specific protein fragment associated with this name or any other molecules that have analogous biological function that share at least about 70%, or alternatively at least about 80% amino acid sequence identity, preferably at least about 90% sequence identity, more preferably at least about 95% sequence identity with the 4-1BB costimulatory signaling region sequence as shown herein. Non-limiting example sequences of the 4-1BB costimulatory signaling region are provided in U.S. Publication 20130266551A1, such as the exemplary sequence provided below: 4-1BB costimulatory signaling region: KRGRKKLLYIFKQPFMRPVQTTQEEDGCSCRFPEEEEGGCEL (SEQ ID NO: 36); and Intracellular domain: 4-1BB co-stimulatory signaling region coding sequence:

(SEQ ID NO: 21)
AAACGGGGCAGAAAGAAACTCCTGTATATATTCAAACAACCATTT
ATGAGACCAGTACAAACTACTCAAGAGGAAGATGGCTGTAGCTGC
CGATTTCCAGAAGAAGAAGAAGGAGGATGTGAACTG.

As used herein, the term “CD28 costimulatory signaling region” or “CD28 costimulatory region” refers to a specific protein fragment associated with this name or any other molecules that have analogous biological function that share at least about 70%, or alternatively at least about 80% amino acid sequence identity, preferably at least about 90% sequence identity, more preferably at least about 95% sequence identity with the CD28 costimulatory signaling region sequence shown herein. The example sequences CD28 costimulatory signaling domain are provided in U.S. Pat. No. 5,686,281; Geiger, T. L. et al., Blood 98: 2364-2371 (2001); Hombach, A. et al., J Immunol 167: 6123-6131 (2001); Maher, J. et al. Nat Biotechnol 20: 70-75 (2002); Haynes, N. M. et al., J Immunol 169: 5780-5786 (2002); or Haynes, N. M. et al., Blood 100: 3155-3163 (2002). Non-limiting examples include residues 114-220 of the below CD28 Sequence: MLRLLLALNL FPSIQVTGNK ILVKQSPMLV AYDNAVNLSC KYSYNLFSRE FRASLHKGLDSAVEVCVVYG NYSQQLQVYS KTGFNCDGKL GNESVTFYLQ NLYVNQTDIY FCKIEVMYPPPYLDNEKSNG TIIHVKGKHL CPSPLFPGPS KPFWVLVVVG GVLACYSLLVTVAFIIFWVR SKRSRLLHSD YMNMTPRRPG PTRKHYQPYA PPRDFAAYRS (SEQ ID NO: 22), and equivalents thereof. In some embodiments, a CD28 costimulatory signaling region comprises, or consists essentially of, or consists of RSKRSRLLHSDYMNMTPRRPGPTRKHYQPYAPPRDFAAYRS (SEQ ID NO: 37) or an equivalent thereof. In further embodiments, a CD28 co-stimulatory signaling region coding sequence comprises, or consists essentially of, or consists of

(SEQ ID NO: 23)
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCC

As used herein, the term “ICOS costimulatory signaling region” or “ICOS costimulatory region” refers to a specific protein fragment associated with this name or any other molecules that have analogous biological function that share at least about 70%, or alternatively at least about 80% amino acid sequence identity, preferably at least about 90% sequence identity, more preferably at least about 95% sequence identity with the ICOS costimulatory signaling region sequence as shown herein. Non-limiting example sequences of the ICOS costimulatory signaling region are provided in U.S. Publication 2015/0017141A1 as well as ICOS costimulatory signaling region coding sequence: ACAAAAAAGA AGTATTCATC CAGTGTGCAC GACCCTAACG GTGAATACAT GTTCATGAGA GCAGTGAACA CAGCCAAAAA ATCCAGACTC ACAGATGTGA CCCTA (SEQ ID NO: 24) or an equivalent thereof.

As used herein, the term “OX40 costimulatory signaling region” or “OX40 costimulatory region” refers to a specific protein fragment associated with this name or any other molecules that have analogous biological function that share at least about 70%, or alternatively at least about 80% amino acid sequence identity, or alternatively at least about 90% sequence identity, or alternatively at least about 95% sequence identity with the OX40 costimulatory signaling region sequence as shown herein. Non-limiting example sequences of the OX40 costimulatory signaling region are disclosed in U.S. Publication 2012/20148552A1, and include the exemplary sequence provided below: OX40 costimulatory signaling region coding sequence: AGGGACCAG AGGCTGCCCC CCGATGCCCA CAAGCCCCCT GGGGGAGGCA GTTTCCGGAC CCCCATCCAA GAGGAGCAGG CCGACGCCCA CTCCACCCTG GCCAAGATC (SEQ ID NO: 25), and equivalents thereof.

As used herein, the term “DAP10 costimulatory signaling region” or “DAP10 costimulatory region” refers to a specific protein fragment associated with this name or any other molecules that have analogous biological function that share at least about 70%, or alternatively at least about 80% amino acid sequence identity, or alternatively at least about 90% sequence identity, or alternatively at least about 95% sequence identity with the DAP10 costimulatory signaling region sequence as shown herein. Non-limiting example sequences of the DAP10 costimulatory signaling region are disclosed in U.S. Pat. No. 9,587,020B2, and include the exemplary sequence: RPRRSPAQDGKVYINMPGRG (SEQ ID NO: 26), or equivalents thereof.

As used herein, the term “DAP12 costimulatory signaling region” or “DAP12 costimulatory region” refers to a specific protein fragment associated with this name or any other molecules that have analogous biological function that share at least about 70%, or alternatively at least about 80% amino acid sequence identity, or alternatively at least about 90% sequence identity, or alternatively at least about 95% sequence identity with the DAP12 costimulatory signaling region sequence as shown herein U.S. Pat. No. 9,587,020B2. Non-limiting example sequences of the DAP12 costimulatory signaling sequence includes the exemplary sequence: ESPYQELQGQRSDVYSDLNTQ (SEQ ID NO: 27), or equivalents thereof.

As used herein, the term “CD3 zeta signaling domain” refers to a specific protein fragment associated with this name or any other molecules that have analogous biological function that share at least about 70%, or alternatively at least about 80% amino acid sequence identity, preferably at least about 90% sequence identity, more preferably at least about 95% sequence identity with the CD3 zeta signaling domain sequence as shown herein. Non-limiting example sequences of the CD3 zeta signaling domain are provided in U.S. Publication 20130266551A1, e.g.: RVKFSRSADAPAYQQGQNQLYNELNLGRREEYDVLDKRRGRDPEMGGKPRRKNP QEGLYNELQKDKMAEAYSEIGMKGERRRGKGHDGLYQGLSTATKDTYDALHMQ ALPPR (SEQ ID NO: 28); and Intracellular domain: CD3 zeta signaling region coding sequence:

(SEQ ID NO: 29)
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGAGAAGGAAGAACCCTCAGGAAGGCCTGTACAAT
GAACTGCAGAAAGATAAGATGGCGGAGGCCTACAGTGAGATTGGG
ATGAAAGGCGAGCGCCGGAGGGGCAAGGGGCACGATGGCCTTTAC
CAGGGTCTCAGTACAGCCACCAAGGACACCTACGACGCCCTTCAC
ATGCAGGCCCTGCCCCCTCGCTAA

In some embodiments, the term “region” and “domain” refer to polypeptide or a fragment thereof and are used interchangeably.

Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein. The cells, polynucleotides, proteins or CAR constructs can be, in some embodiments, detectably labelled.

As used herein, the term “label” or a detectable label intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

As used herein, a purification label or marker refers to a label that may be used in purifying the molecule or component that the label is conjugated to, such as an epitope tag (including but not limited to a Myc tag, a human influenza hemagglutinin (HA) tag, a FLAG tag), an affinity tag (including but not limited to a glutathione-S transferase (GST), a poly-Histidine (His) tag, Calmodulin Binding Protein (CBP), or Maltose-binding protein (MBP)), or a fluorescent tag.

The terms effective period (or time) and effective conditions refer to a period of time or other controllable conditions (e.g., temperature, humidity for in vitro or ex vivo methods), necessary or preferred for an agent or composition to achieve its intended result, e.g., the differentiation or dedifferentiation of cells to a pre-determined cell type.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively more than 95%, of the cells are of the same or similar phenotype. Phenotype can be determined by a pre-selected cell surface marker or other marker.

The terms or “acceptable,” “effective,” or “sufficient” when used to describe the selection of any components, ranges, dose forms, etc. disclosed herein intend that said component, range, dose form, etc. is suitable for the disclosed purpose.

As used herein, the terms “treating,” “treatment” and the like are used herein to mean obtaining a desired pharmacologic or physiologic effect. In some embodiments, the effect can be prophylactic in terms of completely or partially preventing a disorder or sign or symptom thereof, or can be therapeutic in terms of a partial or complete cure for a disorder or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; or relieving or ameliorating the symptoms of disorder. In some embodiments, treatment is the arrestment of the development of symptoms of the disease or disorder, e.g., a cancer. In some embodiments, they refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In some embodiments where the disease is an immune cell cancer, such as multiple myeloma (MM) or an acute myeloid leukemia (AML), reduction in an immunoglobulin (such as IgG) level, or residual cancer cells (for example as measured by flow cytometry, RT-PCR, or other conventional clinical methods), or both, in a biological sample of a subject, such as peripheral blood, plasma or serum, may be used as a clinical end point. In some embodiments where the disease is a cancer or tumor, reduction in circulating tumor cells (CTCs, which refers to a cell that is shed into the vasculature or lymphatics and is carried around the subject body in the blood circulation) in a biological sample of a subject (for example as measured by PCR or other suitable clinical methods), such as peripheral blood, plasma or serum, may be used as a clinical end point. In some embodiments, treatment excludes prophylaxis. In one aspect, treatment excludes prophylaxis.

As used herein, the term “sample” and “biological sample” are used interchangeably, referring to sample material derived from a subject. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples may include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. In some embodiments, a biological sample is selected from peripheral blood, plasma or serum.

As used herein, a therapeutic protein or polypeptide refers to a protein or a polypeptide suitable for a treatment, including but not limited to an antibody or a fragment thereof, an enzyme, a ligand or a receptor. Such therapeutic protein or polypeptide may be chose by a physician or one of skill in the art, based on the disease to be treated. For example, for treating a cancer, an antibody to an immune checkpoint receptor or a ligand thereof may be used, such as an anti-PD-1 antibody or an anti-PD-L1 antibody or both.

As used herein, the term “ligand” refers to any molecule or atom that binds to a receiving protein molecule or receptor. The ligand may be capable of delivering a signal to the cell or cells, or capable of activating various cellular processes.

As used herein, the term “pathogen” refers to an infectious agent capable of causing an infection within a host. Various pathogens may include bacteria, viruses, fungi, protists, parasites or any other microorganism capable of producing a disease. In one embodiment, the term “disease” or “disorder” as used herein refers to a pathogenic infection, a status of being diagnosed with a pathogenic infection, a status of being suspect of having a pathogenic infection, or a status of at high risk of having a pathogenic infection.

In one embodiment, the term “disease” or “disorder” as used herein refers to a cancer, a status of being diagnosed with a cancer, a status of being suspect of having a cancer, or a status of at high risk of having a cancer.

As used herein, a “cancer” is a disease state characterized by the presence in a subject of cells demonstrating abnormal uncontrolled replication and, in some aspects, the term may be used interchangeably with the term “tumor.” The term “cancer or tumor antigen” refers to an antigen known to be associated and expressed on the surface with a cancer cell or tumor cell or tissue, and the term “cancer or tumor targeting antibody” refers to an antibody that targets such an antigen. In some embodiments, the term “cancer” as used herein refers to multiple myeloma (MM). In some embodiments, the term “cancer” as used herein refers to acute myeloid leukemia (AML). Additionally or alternatively, the cancer as used herein expresses one or more of CD19, mesothelin, ROR1, or EGFRVIII. In some embodiment, the cancer is a relapsed cancer. In some embodiments, the cancer is a refractory cancer.

Mesothelin is a membrane-anchored preproprotein that plays a role in cell division. Additionally, Mesothelin is a megakaryocyte-potentiating factor that functions as a cytokine that can stimulate colony formation of bone marrow megekaryocites. Mesothelin is overexpressed in epithelial mesotheliomas, pancreatic, ovarian cancers and in specific squamous cell carcinomas. Anti-mesothelin antibodies are known in the art, and are described in Hassan et al., Clin. Cancer Res., Dec. 15 2010 (16)(24) 6132-6138. Mesothelin targeting CARs are known in the art, see, e.g., U.S. Pat. Nos. 7,592,426 and 9,023,351; Castelletti et al. (2021) Biomark Res. February 15; 9(1):11 and are commercially available from Creative Biolabs (see https://www.creative-biolabs.com/car-t/target-mesothelin-69.htm, accessed on Sep. 17, 2021).

ROR1 is a glycosylated type-I membrane receptor tyrosine kinase-like orphan receptor protein. Increased expression of ROR1 is associated with B-cell chronic lymphocytic leukemia, lung cancer, breast cancer and ovarian cancer. Anti-ROR1 antibodies are known in the art, see e.g., Choi et al. (2015) Blood: 126(23):1736 and a Fab fragment Yin et al. (2017) Oncotarget November 7 *(55):94210-94222. Anti-ROR1 CARs are known in the art, see, e.g., U.S. Patent Publications US20180142016A1; US20130251723A1, Wallstabe et al. (2019) JCI Insights, September 19; 4(18)e126345 and Prussak et al., https://www.oncternal.com/_documents/ASCO%20SITC%202020%20ROR1%20CAR-T%20poster_Final.pdf.

Epidermal growth factor receptor variant III (EGFRvIII) is an epidermal growth factor receptor including a deletion of exons 2-7 of the EGFR gene and renders the mutant receptor incapable of binding any known ligand. Despite this, EGFRvIII displays low-level constitutive signaling that is augmented by reduced internalization and downregulation. Aberrant EGFRvIII signaling has been shown to be important in driving tumor progression and often correlates with poor prognosis. (See Gan H. K. et al., (2013) FEBS J., November; 280(21):5350-70. doi: 10.1111/febs.12393. Epub 2013 Jul. 8.) Anti-EGFRvIII antibodies are known in the art, see, e.g., U.S. Pat. No. 10,221,242.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active polypeptide, polynucleotide, antibody, or cell with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton). The term pharmaceutically acceptable carrier (or medium), which may be used interchangeably with the term biologically compatible carrier or medium, refers to reagents, cells, compounds, materials, compositions, or dosage forms, or any combination thereof, that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit to risk ratio. Pharmaceutically acceptable carriers suitable for use in the present disclosure include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodible). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result or protection or both desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

As used herein, the term “contacting” means direct or indirect binding or interaction between two or more molecules or other entities. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” or “delivery” of a cell or vector or other agent and compositions containing same can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the administration is an intratumoral administration, or administration to a tumor microenvironment, or both. In some embodiments, the administration is an infusion (for example to peripheral blood of a subject) over a certain period of time, such as about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 24 hours or longer.

The term administration shall include without limitation, administration by oral, parenteral (e.g., intramuscular, intraperitoneal, intravenous, intracerebroventricular (ICV), intrathecal, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The disclosure is not limited by the route of administration, the formulation or dosing schedule.

“Administration” can be performed in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. In some embodiments, 1×10⁴ to 1×10¹⁵ or ranges in between of cells as disclosed herein are administrated to a subject, such as 1×10⁷ to 1×10¹⁰. In some embodiments, administering or a grammatical variation thereof also refers to more than one doses with certain interval. In some embodiments, the interval is 1 day, 2 days, 3 days, 4 days, 5 days, 6 days, 1 week, 10 days, 2 weeks, 3 weeks, 1 month, 2 months, 3 months, 4 months, 5 months, 6 months, 1 year or longer. In some embodiments, one dose is repeated for once, twice, three times, four times, five times, six times, seven times, eight times, nine times, ten times or more. For example, cells as disclosed herein may be administered to a subject weekly and for up to four weeks. The compositions and therapies can be combined with other therapies, e.g., lymphodepletion chemotherapy followed by infusions (e.g., four weekly infusions) of the therapy, defining one cycle, followed by additional cycles until a partial or complete response is seen or alternatively utilized as a “bridging” therapy to another modality, such as hematopoietic stem cell transplantation or CAR T cell therapy.

An agent of the present disclosure can be administered for therapy by any suitable route of administration. It will also be appreciated that the optimal route will vary with the condition and age of the recipient, and the disease being treated.

A “subject,” “individual” or “patient” is used interchangeably herein, and refers to a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present disclosure is also useful for veterinary treatment of companion mammals, exotic animals and domesticated animals, including mammals, rodents. In one embodiment, the mammals include horses, dogs, and cats. In another embodiment of the present disclosure, the human is a fetus, an infant, a pre-pubescent subject, an adolescent, a pediatric patient, or an adult. In one aspect, the subject is pre-symptomatic mammal or human. In another aspect, the subject has minimal clinical symptoms of the disease. The subject can be a male or a female, adult, an infant or a pediatric subject. In an additional aspect, the subject is an adult. In some instances, the adult is an adult human, e.g., an adult human greater than 18 years of age.

The term “suffering” as it related to the term “treatment” refers to a patient or individual who has been diagnosed with or is predisposed to a disease as disclosed herein. This patient has not yet developed characteristic disease pathology.

As noted herein, an “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro, or ex vivo, or in vivo tests (or any combination thereof) initially can provide useful guidance on the proper doses for patient administration. In general, one will desire to administer an amount of the agent as disclosed herein (such as a cell) that is effective to achieve a serum level commensurate with the concentrations found to be effective in vitro or ex vivo. Determination of these parameters is well within the skill of the art. These considerations, as well as effective formulations and administration procedures are well known in the art and are described in standard textbooks.

“Therapeutically effective amount” of a drug or an agent refers to an amount of the drug or the agent (such as a cell as disclosed herein) that is an amount sufficient to obtain a pharmacological response; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses, as needed to induce a partial or complete effect. Thus, a therapeutically effective amount may be administered in one or more administrations. In some embodiments, a therapeutically effective amount of cells as disclosed herein is 1×10⁴ to 1×10¹⁵ or ranges, such as 1×10⁷ to 1×10¹⁰.

In some embodiments, a treatment, such as an immune cell comprising a polypeptide as disclosed herein, is administered to a subject as disclosed herein in an effective amount. In further embodiments, a treatment, such as an immune cell comprising a polypeptide as disclosed herein, is administered to a subject as disclosed herein in a therapeutically effective amount.

An “anti-cancer therapy,” as used herein, includes but is not limited to surgical resection, chemotherapy, cryotherapy, radiation therapy, immunotherapy and targeted therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites.

Topoisomerase inhibitors are drugs that interfere with the action of topoisomerase enzymes (topoisomerase I and II). During the process of chemo treatments, topoisomerase enzymes control the manipulation of the structure of DNA necessary for replication and are thus cell cycle specific. Examples of topoisomerase I inhibitors include the camptothecan analogs listed above, irinotecan and topotecan. Examples of topoisomerase II inhibitors include amsacrine, etoposide, etoposide phosphate, and teniposide.

Antimetabolites are usually analogs of normal metabolic substrates, often interfering with processes involved in chromosomal replication. They attack cells at very specific phases in the cycle. Antimetabolites include folic acid antagonists, e.g., methotrexate; pyrimidine antagonist, e.g., 5-fluorouracil, floxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and teniposide; and camptothecan analogs, e.g., irinotecan and topotecan.

In some embodiments where the cancer is an immune cell cancer, an anti-cancer therapy may comprise, or consist essentially of, or consist of a hematopoietic stem cell transplantation.

In some embodiments, a therapeutic agent, such as a cell as disclosed herein, may be combined in treating a cancer with another anti-cancer therapy or a therapy depleting an immune cell. For example, lymphodepletion chemotherapy is performed followed by administration of a cell as disclosed herein, such as four weekly infusions. In further embodiments, these steps may be repeated for once, twice, three or more times until a partial or complete effect is observed or a clinical end point is achieved.

Cryotherapy includes, but is not limited to, therapies involving decreasing the temperature, for example, hypothermic therapy.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy or a dose of ultraviolet radiation at a range from at least about 5 J/m² to not more than about 50 J/m², usually about 10 J/m².

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition”. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

### MODES FOR CARRYING OUT THE DISCLOSURE

### Engineered Immune Cells

In one aspect, provided herein is an immune cell engineered to decrease expression and/or function of RelB in the engineered immune cell. In a further aspect, provided herein is an engineered immune cell engineered to decrease expression and/or function of RelB in the immune cell. As used herein, the expression and/or function of the RelB is decrease as compared to a native immune cell or non-engineered cell. One can determine if RelB is decrease by detecting the level or amount of RelB mRNA or protein expressed by the cell using methods known in the art and described herein. One can also screen or assay for reduced expression of PD-1, TIM3, LAG3, TIGIT and 2B4 in the engineered cells as compared to cells without the RelB modification and/or increased expression of CD44, a marker of activated CD8 T cells; higher levels of the cytokines TNF and IFN-gamma after stimulation, and higher levels of several markers of effector CD8 T cells (KLRG1, granzyme B, CD107alpha or “CD107a”).

The immune cell for making the engineered immune cell can be a primary immune cell or can be a cultured immune cell. Non-limiting examples of immune cells include, e.g., white blood cells (leukocytes, such as granulocytes (neutrophils, eosinophils, and basophils), macrophages, monocytes, and lymphocytes (T cells, B cells, natural killer (NK) cells and NKT cells)) which may be derived from hematopoietic stem cells (HSCs) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSCs), hematopoietic stem cells (HSCs), or immortalized cells. In some embodiments, the HSCs are derived from umbilical cord blood of a subject, peripheral blood of a subject, or bone marrow of a subject.

In one embodiment, the immune cell that is used for the production of the engineered immune cell is from the group of a T cell, a CD4⁺ T cell, a CD8⁺ T cell, a macrophage, a stem cell or a Natural Killer (NK) T cell. In a particular aspect, the immune cell is a T cell, optionally a CD4⁺ T cell or a CD8⁺ T cell. The immune cell can be of any appropriate animal or mammalian species, e.g., canine, feline, equine, murine, rat or human.

In one embodiment, RelB function or expression in the engineered immune cell by incorporation of a vector expressing a polynucleotide encoding the RelB that is transduced into the immune cell to produce the engineered immune cell. Polynucleotides encoding RelB proteins are known in the art and described herein. Examples of such include polynucleotides encoding all isoforms, including the following RelB isoform 1, RelB isoform 2, and RelB isoform 3:

RelB Amino Acid Sequence (Human, (SEQ ID NO: 1):
MLRSGPASGPSVPTGRAMPSRRVARPPAAPELGALGSPDLSSLSL
AVSRSTDELEIIDEYIKENGFGLDGGQPGPGEGLPRLVSRGAASL
STVTLGPVAPPATPPPWGCPLGRLVSPAPGPGPQPHLVITEQPKQ
RGMRFRYECEGRSAGSILGESSTEASKTLPAIELRDCGGLREVEV
TACLVWKDWPHRVHPHSLVGKDCTDGICRVRLRPHVSPRHSFNNL
GIQCVRKKEIEAAIERKIQLGIDPYNAGSLKNHQEVDMNVVRICF
QASYRDQQGQMRRMDPVLSEPVYDKKSTNTSELRICRINKESGPC
TGGEELYLLCDKVQKEDISVVFSRASWEGRADFSQADVHRQIAIV
FKTPPYEDLEIVEPVTVNVFLQRLTDGVCSEPLPFTYLPRDHDSY
GVDKKRKRGMPDVLGELNSSDPHGIESKRRKKKPAILDHFLPNHG
SGPFLPPSALLPDPDFFSGTVSLPGLEPPGGPDLLDDGFAYDPTA
PTLFTMLDLLPPAPPHASAVVCSGGAGAVVGETPGPEPLTLDSYQ
APGPGDGGTASLVGSNMFPNHYREAAFGGGLLSPGPEAT
disclosed in UniProtKB/Swiss-Prot Ref.
Sequence: Q01201.
Examples of RelB isoforms include the following
amino acid sequences:
(SEQ ID NO: 32):
MLRSGPASGPSVPTGRAMPSRRVARPPAAPELGALGSPDLSSLSLA
VSRSTGSSSTDA.
Disclosed in UniProtKB/Swiss-Prot Ref.
Sequence: D6RIV7.
(SEQ ID NO: 33):
MLRSGPASGPSVPTGRAMPSRRVARPPAAPELGALGSPDLSSLSL
AVSRSTEIIDEYIKENGFGLDGGQPGPGEGLPRLVSRGAASLSTV
TLGPVAPPATPPPWGCPLGRLVSPAPGPGPQPHLVITEQPKQRGM
RFRYECEGRSAGSILGESSTEASKTLPAIELRDCGGLREVEVTAC
LVWKDWPHRVHPHSLVGKDCTDGICRVRLRPHVSPRHSFNNLGIQ
CVRKKEIEAAIERKIQLGIDPYNAGSLKNHQEVDMNVVRICFQAS
YRDQQGQMRRMDPVLSEPVYDKKSTNTSELRICRINKESGPCTGG
EELYLLCDKVQKEDISVVFSRASWEGRADFSQADVHRQIAIVFKT
PPYEDLEIVEPVTVNVFLQRLTDGVCSEPLPFTYLPRDHDSYGVD
KKRKRGMPDVLGELNSSDPHGIESKRRKKKPAILDHFLPNHGSGP
FLPPSALLPDPDFFSGTVSLPGLEPPGGPDLLDDGFAYDPTAPTL
FTMLDLLPPAPPHASAVVCSGGAGAVVGETPGPEPLTLDSYQAPG
PGDGGTASLVGSNMFPNHYREAAFGGGLLSPGPEAT.
Disclosed in UniProtKB/Swiss-Prot Ref.
Sequence: D6RIV7.
(SEQ ID NO: 34):
EIIDEYIKENGFGLDGGQPGPGEGLPRLVSRGAASLSTVTLGPVA
PPATPPPWGCPLGRLVSPAPGPGPQPHLVITEQPKQRGMRFRYEC
EGRSAGSILGESSTEASKTLPAIEF.
Disclosed in UniProtKB/Swiss-Prot Ref.
Sequence: A0A8V8TQY2.
(SEQ ID NO: 35):
XSEPLPFTYLPRDHGPFLPPSALLPDPDFFSGTVSLPGLEPPGGP
DLLDDGFAYDPTAPTLFTMLDLLPPAPPHASAVVCSGGAGAVVGE
TPGPEPLTLDSYQAPGPGDGGTASLVGSNMFPNHYREAAFGGGLL
SPGPEAT.
Disclosed in UniProtKB/Swiss-Prot Ref.
Sequence: K7ERX9.

To express the RelB in the immune cell, the polynucleotide can be contained within an expression vector and operatively linked to regulatory elements, such as a promoter and/or enhancer to facilitate expression. In some embodiments, the coding polynucleotide is introduced to the cell population via a vector. In further embodiments, the vector is a viral vector or a non-viral vector. In some embodiments, the non-viral vector is a plasmid. In some embodiments, the viral vector is selected form a retroviral vector, a lentiviral vector, an adenoviral vector, an adeno-associated viral vector or Herpes viral vector. In a further embodiment, the viral backbone contains essential nucleic acids or sequences for integration of the coding polynucleotide into a target cell's genome. In some embodiments, the essential nucleic acids necessary for integration to the genome of the target cell include at the 5′ and 3′ ends the minimal LTR regions required for integration of the vector.

This disclosure also provides a vector comprising, or alternatively consisting essentially of, or yet further consisting of a polynucleotide encoding a RelB protein (such as coding polynucleotide) as disclosed herein, optionally inserted into a viral backbone. In some embodiments, the vector is selected for expression in prokaryotic or eukaryotic cells. In some embodiments, the vector comprises, or alternatively consists essentially of, or yet further consists of a polynucleotide as described herein, encoding the modified protein. In some embodiments, the vector comprises, or alternatively consists essentially of, or yet further consists of a polynucleotide as described herein, permitting replication of the polynucleotide. In further embodiments, the vector further comprises a regulatory sequence operatively linked to the polynucleotide and directing the replication of the polynucleotide. In yet a further embodiment, the regulatory sequence comprises, or alternatively consists essentially of, or yet further consists of one or more of: a promoter, an intron, an enhancer, a polyadenylation signal, a terminator, a silencer, a TATA box, or a Woodchuck Hepatitis Virus (WHP) Posttranscriptional Regulatory Element (WPRE).

In a further aspect, the engineered immune cell also expresses a receptor or ligand that binds at least one tumor antigen or at least one antigen expressed by a pathogen. The receptor or ligand can be a naturally occurring or the immune cell can be engineered to express the receptor or ligand that binds tumor antigen or the antigen expressed by the pathogen. Non-limiting examples of tumor antigens are selected from the group of an antigenic substance of a cancer or tumor cells. In some embodiments, a tumor antigen presents on some tumor or cancer cells and also on some normal cells, optionally at a lower level. In some embodiments, the tumor antigen only presents on a tumor or cancer cell but not on a normal cell. In some embodiments, the tumor antigen is selected from G Protein-Coupled Receptor Class C Group 5 Member D (GPRC5D), B-cell maturation antigen (BCMA), SLAMF7 (CS1 or CD319), EGFR, wildtype epidermal growth factor receptor (EGFRwt), epidermal growth factor receptor variant III (EGFRVIII), FLT3, CD70, mesothelin, CD123, CD19, carcinoembryonic antigen (CEA), CD133, human epidermal growth factor receptor 2 (HER2), ERBB2 (Her2/neu), CD22, CD30, CD171, CLL-1 (CLECL1), GTPase-activating protein (GAP), CD5, interleukin 13 receptor alpha 2 (IL13Ra2), guanylyl cyclase C (GUCY2C), tumor-associated glycoprotein-72 (TAG-72), thymidine kinase 1 (TK1), hypoxanthine guanine phosphoribosyltransferase (HPRT1), cancer/testis (CT), CD33, ganglioside G2 (GD2), GD3, Tn Ag, prostate specific membrane antigen (PSMA), receptor tyrosine kinase-like orphan receptor 1 (ROR1), TAG72, CD38, CD44v6, epithelial cell adhesion molecule precursor (EpCam or EPCAM), B7H3, KIT, IL-13Ra2, IL-11Rα, prostate stem cell antigen (PSCA), PRSS21, vascular endothelial growth factor receptor 2 (VEGFR2), LewisY, CD24, PDGFR-beta, SSEA-4, CD20, Folate receptor alpha, mucin 1 (Muc1), NCAM, Prostase, PAP, ELF2M, Ephrin B2, fibroblast activation protein alpha (FAP), IGF-I receptor, CAIX, LMP2, gp100, bcr-abl, tyrosinase, ephrin type-A receptor 2 precursor (EphA2), Fucosyl GM1, sLe, GM3, TGS5, HMWMAA, o-acetyl-GD2, Folate receptor beta, TEM1/CD248, TEM7R, CLDN6, TSHR, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, legumain, HPV E6, E7, intestinal carboxyl esterase, mut hsp70-2, CD79a, CD79b, CD72, LAIR1, FCAR, LILRA2, CD300LF, CLEC12A, BST2, EMR2, LY75, glypican 3 (GPC3), FCRL5, or IGLL1.

In specific embodiments, the tumor or cancer antigen is from: CD19, mesothelin, ROR1, or EGFRvIII.

In one aspect, the receptor or ligand is an antibody that binds the tumor, such as an anti-CD19 antibody, anti-mesothelin antibody, anti-ROR1 antibody, or anti-EGFRvIII antibody or an antigen binding fragment thereof, e.g., a scFv fragment or a fragment comprising at least the six CDRs or the heavy and light chains of the reference antibody.

When the engineered immune cell expresses a ligand or receptor that binds an antigen expressed by the pathogen, the antigen expressed by the pathogen includes for example, an antigen expressed in a virus and/or encoded by a viral genome. Non-limiting example includes hemagglutinin (HA) and neuraminidase (NA) of an influenza virus, and spike protein, S1, S2, nucleocapsid envelope protein of a COVID-19.

In one aspect, the engineered immune cell further comprises a suicide gene. In further embodiments, the suicide gene product is selected from one or more of: HSV-TK (Herpes simplex virus thymidine kinase), cytosine deaminase, nitroreductase, carboxylesterase, cytochrome P450 or PNP (Purine nucleoside phosphorylase), truncated EGFR, or inducible caspase (“iCasp”). In some embodiments, the coding polynucleotide further comprises a regulatory sequence directing expression of the suicide gene. In yet further embodiments, the regulatory sequence is inducible.

### CAR-Expressing Engineered Immune Cells

In one aspect, the receptor or ligand is expressed in the engineered immune cell by introduction of a polynucleotide encoding a chimeric antigen receptor (CAR) and thus the immune cell further comprises a CAR. Thus, this disclosure also provides an engineered immune cell as described above that further comprises a CAR that bind to a cancer or tumor antigen or a pathogenic antigen, the CAR comprising, or consisting essentially of, or consisting of, antigen binding domain, transmembrane, and intracellular domain. The intracellular domain or cytoplasmic domain comprises a costimulatory signaling region and a zeta chain portion. The CAR may optionally further comprise a spacer domain of up to 300 amino acids, preferably 10 to 100 amino acids, more preferably 25 to 50 amino acids.

Spacer Domain. The CAR may optionally further comprise a spacer or linker domain of up to 300 amino acids, preferably 10 to 100 amino acids, more preferably 25 to 50 amino acids. For example, the spacer may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. A spacer domain may comprise, for example, a portion of a human Fc domain, a CH3 domain, or the hinge region of any immunoglobulin, such as IgA, IgD, IgE, IgG, or IgM, or variants thereof. For example, some embodiments may comprise an IgG4 hinge with or without a S228P, L235E, and/or N297Q mutation (according to Kabat numbering). Additional spacers include, but are not limited to, CD4, CD8, and CD28 hinge regions.

Antigen Binding Domain. In certain aspects, the present disclosure provides a CAR that comprises, or alternatively consists essentially thereof, or yet further consists of an antigen binding domain specific to a cancer antigen, tumor antigen or antigen expressed by a pathogen. Examples of such are described above. The antigen binding domains can be from any appropriate species, e.g., murine, human or a humanized sequence. The antigen binding domain comprises, or alternatively consists essentially thereof, or yet consists of the antigen binding domain of an anti-cancer, tumor or pathogen antibody. Monoclonal antibodies that specifically bind to target antigens are commercially available. The antigen binding domains can be from any appropriate species, e.g., murine, human or a humanized sequence. In one aspect, the antigen binding domain comprises the six CDRs of the antibody or the heavy chain variable region and the light chain variable region of an antibody or an equivalent of thereof), for example, an scFv. An scFv region can comprise the variable regions of the heavy (V_{H}) and light chains (V_{L}) of immunoglobulins, connected with a short linker peptide, e.g., of the sequence (GGGGS)n wherein n is an integer from 1 to 6 (SEQ ID NO: 38). The linker peptide may be from 1 to 50 amino acids, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, the linker is glycine rich, although it may also contain serine or threonine.

In some embodiments, the antigen binding domain comprises, or alternatively consists essentially thereof, or yet consists of the antigen binding domain of an anti-CD19antibody or an antibody that binds CD19. Monoclonal antibodies that specifically bind CD19 are commercially available. The antigen binding domains can be from any appropriate species, e.g., murine, human or a humanized sequence. In one aspect, the antigen binding domain comprises the six CDRs of the antibody or the heavy chain variable region and the light chain variable region of an antibody to CD19 or an equivalent of thereof), for example, an scFv. An scFv region can comprise the variable regions of the heavy (V_{H}) and light chains (V_{L}) of immunoglobulins, connected with a short linker peptide, e.g., of the sequence (GGGGS)n wherein n is an integer from 1 to 6 (SEQ ID NO: 38). The linker peptide may be from 1 to 50 amino acids, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, the linker is glycine rich, although it may also contain serine or threonine.

In some embodiments, the antigen binding domain comprises, or alternatively consists essentially thereof, or yet consists of the antigen binding domain of an anti-BCMA antibody or an antibody that binds a BCMA-relevant antigen. Monoclonal antibodies that specifically bind this antigen are commercially available. The antigen binding domains can be from any appropriate species, e.g., murine, human or a humanized sequence. In one aspect, the antigen binding domain comprises the heavy chain variable region and the light chain variable region of an antibody to B-cell maturation antigen (BCMA) or an equivalent of thereof), for example, an scFv. An scFv region can comprise the variable regions of the heavy (V_{H}) and light chains (V_{L}) of immunoglobulins, connected with a short linker peptide e.g., of the sequence (GGGGS)n wherein n is an integer from 1 to 6 (SEQ ID NO: 38). The linker peptide may be from 1 to 50 amino acids, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, the linker is glycine rich, although it may also contain serine or threonine.

In some embodiments, the antigen binding domain comprises, or alternatively consists essentially thereof, or yet consists of the antigen binding domain of an anti-ROR1 antibody or an antibody that binds an ROR1-relevant antigen. Monoclonal antibodies that specifically binds this antigen are commercially available. The antigen binding domains can be from any appropriate species, e.g., murine, human or a humanized sequence. In one aspect, the antigen binding domain comprises the heavy chain variable region and the light chain variable region of an antibody to ROR1 and/or an equivalent of thereof), for example, an scFv. An scFv region can comprise the variable regions of the heavy (V_{H}) and light chains (V_{L}) of immunoglobulins, connected with a short linker peptide e.g., of the sequence (GGGGS)n wherein n is an integer from 1 to 6 (SEQ ID NO: 38). The linker peptide may be from 1 to 50 amino acids, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, the linker is glycine rich, although it may also contain serine or threonine.

In some embodiments, the antigen binding domain comprises, or alternatively consists essentially thereof, or yet consists of the antigen binding domain of an anti-EGFRvIII antibody or an antibody that binds an EGFRvIII-relevant antigen. Monoclonal antibodies that specifically binds this antigen are commercially available. The antigen binding domains can be from any appropriate species, e.g., murine, human or a humanized sequence. In one aspect, the antigen binding domain comprises the heavy chain variable region and the light chain variable region of an antibody to EGFRvIII or an equivalent of thereof), for example, an scFv. An scFv region can comprise the variable regions of the heavy (V_{H}) and light chains (V_{L}) of immunoglobulins, connected with a short linker peptide e.g., of the sequence (GGGGS)n wherein n is an integer from 1 to 6 (SEQ ID NO: 38). The linker peptide may be from 1 to 50 amino acids, for instance, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids. In some embodiments, the linker is glycine rich, although it may also contain serine or threonine.

In another aspect of the present disclosure, the antigen binding domain of a cancer, tumor or pathogenic antibody includes one or more of the following characteristics:
(a) the light chain immunoglobulin variable domain sequence comprises one or more CDRs that are at least 80% identical to a CDR of a light chain variable domain of any of the disclosed light chain sequences;
(b) the heavy chain immunoglobulin variable domain sequence comprises one or more CDRs that are at least 80% identical to a CDR of a heavy chain variable domain of any of the disclosed heavy chain sequences;
(c) the light chain immunoglobulin variable domain sequence is at least 80% identical to a light chain variable domain of any of the disclosed light chain sequences;
(d) the HC immunoglobulin variable domain sequence is at least 80% identical to a heavy chain variable domain of any of the disclosed light chain sequences; and
(e) the antibody binds an epitope that overlaps with an epitope bound by any of the disclosed sequences.

Additional examples of equivalents include peptide having at least 85%, or alternatively at least 90%, or alternatively at least 95%, or alternatively at least 97% amino acid identity to the peptide or a polypeptide that is encoded by a polynucleotide that hybridizes under conditions of high stringency to the complement of a polynucleotide encoding the antigen binding domain, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

Transmembrane Domain. The CAR can contain one or more transmembrane domains that can be derived either from a natural or from a synthetic source. Where the source is natural, the domain may be derived from any membrane-bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Cytoplasmic Domain. The cytoplasmic domain or intracellular signaling domain of the CAR is responsible for activation of at least one of the traditional effector functions of an immune cell in which a CAR has been placed. The intracellular signaling domain refers to a portion of a protein which transduces the effector function signal and directs the immune cell to perform its specific function. An entire signaling domain or a truncated portion thereof may be used so long as the truncated portion is sufficient to transduce the effector function signal. Cytoplasmic sequences of the TCR and co-receptors as well as derivatives or variants thereof can function as intracellular signaling domains for use in a CAR. Intracellular signaling domains of particular use in this disclosure may be derived from FcR, TCR, CD3, CDS, CD22, CD79a, CD79b, CD66d. In some embodiments, the signaling domain of the CAR can comprise a CD3 ζ signaling domain.

Since signals generated through the TCR are alone insufficient for full activation of a T cell, a secondary or co-stimulatory signal may also be required. Thus, the intracellular region of a co-stimulatory signaling molecule, including but not limited the intracellular domains of the proteins CD27, DAP10, DAP12, CD28, 4-IBB (CD 137), OX40, CD30, CD40, PD-1, ICOS, lymphocyte function-associated antigen-1 (LFA-1), CD2, CD7, LIGHT, NKG2C, B7-H3, or a ligand that specifically binds with CD83, may also be included in the cytoplasmic domain of the CAR. For instance, a CAR may comprise one, two, or more co-stimulatory domains, in addition to a signaling domain (e.g., a CD3 ζ signaling domain).

In some embodiments, the cell activation moiety of the chimeric antigen receptor is a T-cell signaling domain comprising, or alternatively consisting essentially of, or yet further consisting of, one or more proteins or fragments thereof selected from the group consisting of CD8 protein, CD28 protein, DAP10, DAP12, 4-1BB protein, OX40, CD30, CD40, PD-1, ICOS, LFA-1, CD2, CD7, CD27, LIGHT, NKG2C, B7-H3, and CD3-zeta protein.

In some embodiments, the cell activation moiety of the chimeric antigen receptor is a T-cell signaling domain comprising, or alternatively consisting essentially of, or yet further consisting of, one or more proteins or fragments thereof selected from the group consisting of CD8 protein, CD28 protein, 4-1BB protein, and CD3-zeta protein.

In specific embodiments, the CAR comprises, or alternatively consists essentially thereof, or yet consists of an antigen binding domain of a cancer, tumor or pathogen targeting antibody, a CD8 α hinge domain, a CD8 α transmembrane domain, a costimulatory signaling region, and a CD3 zeta signaling domain. In further embodiments, the costimulatory signaling region comprises either or both a CD28 costimulatory signaling region and a 4-1BB costimulatory signaling region. In one aspect, the antigen binding domain selectively binds an antigen selected from CD19, BCMA, ROR1 or EGFRVIII.

In one aspect, the CAR of the engineered immune cell comprises a transmembrane domain selected from a CD28 or a CD8 α transmembrane domain; an intracellular domain that comprises one or more costimulatory regions selected from a CD28 costimulatory signaling region, a 4-1BB costimulatory signaling region, an ICOS costimulatory signaling region, a DAP10 costimulatory region, a DAP 12 costimulatory region, or an OX40 costimulatory region; and optionally further comprising a CD3 zeta signaling domain. In one aspect, the antigen binding domain selectively binds an antigen selected from CD19, BCMA, ROR1 or EGFRVIII.

In a further aspect, the CAR is an anti-CD19 CAR of the sequence: 5′-MALPVTALLLPLALLLHAARPEQKLISEEDLDIQMTQTTSSLSASLGDRVTISCRASQ DISKYLNWYQQKPDGTVKLLIYHTSRLHSGVPSRFSGSGSGTDYSLTISNLEQEDIAT YFCQQGNTLPYTFGGGTKLEITGGGGSGGGGSGGGGSEVKLQESGPGLVAPSQSLS VTCTVSGVSLPDYGVSWIRQPPRKGLEWLGVIWGSETTYYNSALKSRLTIIKDNSKS QVFLKMNSLQTDDTAIYYCAKHYYYGGSYAMDYWGQGTSVTVSSIEFMYPPPYL DNERSNGTIIHIKEKHLCHTQSSPKLFWALVVVAGVLFCYGLLVTVALCVIWTNSR RNRGGQSDYMNMTPRRPGLTRKPYQPYAPARDFAAYRPRAKFSRSAETAANLQDP NQLYNELNLGRREEYDVLEKKRARDPEMGGKQQRRRNPQEGVYNALQKDKMAE AYSEIGTKGERRRGKGHDGLYQGLSTATKDTYDALHMQTLAPR-3′ (SEQ ID NO: 30), or an equivalent thereof that binds CD19.

In some embodiments, the CAR can further comprise a detectable marker or purification marker.

Switch Mechanisms. In some embodiments, the CAR may also comprise a switch mechanism for controlling expression and/or activation of the CAR. For example, a CAR may comprise, consist, or consist essentially of an extracellular, transmembrane, and intracellular domain, in which the extracellular domain comprises a target-specific binding element that binds a label, binding domain, or tag that is specific for a molecule other than the target antigen that is expressed on or by a target cell. In such embodiments, the specificity of the CAR is provided by a second construct that comprises, consists, or consists essentially of a target antigen binding domain and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but it cannot bind to a target antigen (i.e., BCMA) until the second composition comprising an BCMA-specific binding domain is administered.

CARs of the present disclosure may likewise require multimerization in order to active their function (see, e.g., US 2015/0368342, US 2016/0175359, US 2015/0368360) and/or an exogenous signal, such as a small molecule drug (US 2016/0166613, Yung et al., Science, 2015) in order to elicit a T-cell response.

Furthermore, the disclosed CARs can comprise a “suicide switch” (also referred to as a “suicide gene”) to induce cell death of the CAR cells following treatment (Buddee et al., PLoS One, 2013) or to downregulate expression of the CAR following binding to the target antigen (WO 2016/011210). A non-limiting exemplary suicide switch or suicide gene is iCasp.

Also provided herein are engineered immune cells comprising the polynucleotides encoding RelB and/or a CAR as described above. The nucleic acids can further comprise the necessary regulatory sequences, e.g., a promoter for expression in a host cell, e.g., a mammalian or human immune or host cell such as a T cell. In one aspect the promoter is a CMV, MND, or an EF1alpha promoter. In a further aspect, the CAR polynucleotide further comprises a marker peptide (e.g., GFP) that may be regulated from a second promoter element, e.g., CMV, MND, and EF1A promoters, located 5′ to the encoding polynucleotide. In one aspect, the second promoter comprises an EF1 alpha promoter. As is apparent to the skilled artisan, the promoter(s) are selected for the host expression system and will vary with the host and the expression vector and intended use.

In a further embodiment, the polynucleotide further comprises a self-cleaving peptide, e.g., a T2A encoding polynucleotide sequence located upstream of the polynucleotide encoding the antigen binding domain.

The polynucleotide can be inserted into an expression vector, e.g., a viral vector, an adenoviral vector, a plasmid, a lentiviral vector or retroviral vector (between the 5′ and 3′ LTRs) or any other vector that can express a gene.

In one aspect, the polynucleotide further comprises a purification marker or detectable label.

An exemplary polynucleotide encoding an anti-CD19 CAR has the sequence: 5′-ATGGCTTTGCCAGTGACAGCTCTTCTCCTTCCACTGGCCCTCCTCCTTCACGCCG CTAGGCCAGAGCAGAAACTTATTTCAGAGGAAGACCTGGACATTCAAATGACA CAAACTACTTCTTCTCTCTCCGCCTCACTTGGTGACCGCGTCACTATTAGTTGCC GCGCTAGTCAAGATATTAGTAAGTACCTGAATTGGTATCAACAAAAACCTGACG GGACTGTAAAGCTGCTTATATATCATACTTCTAGGCTGCATTCTGGAGTACCTTC ACGATTTAGCGGTAGCGGATCCGGCACCGACTACTCCCTCACAATTAGCAATCT GGAGCAAGAGGACATAGCCACCTACTTCTGCCAGCAAGGGAATACCTTGCCAT ACACTTTCGGTGGTGGAACTAAGCTCGAAATTACTGGGGGTGGAGGCAGTGGC GGAGGGGGGTCAGGTGGGGGAGGTTCAGAAGTCAAACTCCAGGAATCTGGACC TGGACTCGTTGCCCCTTCCCAATCCCTTAGTGTTACATGCACTGTATCAGGTGTA TCCCTCCCTGATTACGGTGTCTCCTGGATTCGGCAGCCTCCTCGGAAGGGTCTCG AGTGGTTGGGAGTGATTTGGGGGTCTGAAACTACTTATTATAACAGTGCCCTTA AGAGTAGATTGACTATAATTAAGGATAACAGTAAGTCACAAGTATTCCTCAAA ATGAATTCCTTGCAAACAGACGATACAGCAATATATTACTGCGCAAAACACTAC TACTATGGCGGTAGTTACGCTATGGACTATTGGGGTCAAGGAACCTCTGTCACA GTTTCTAGCATTGAGTTCATGTATCCCCCACCTTACTTGGACAATGAAAGGTCTA ATGGGACCATCATACACATTAAAGAGAAACACCTGTGTCATACTCAGAGTTCTC CAAAATTGTTCTGGGCCTTGGTTGTCGTTGCCGGCGTACTGTTCTGTTACGGTCT CTTGGTTACCGTGGCACTTTGTGTTATCTGGACTAATTCCCGGCGGAATCGGGG TGGACAGAGCGATTACATGAATATGACCCCAAGAAGACCTGGACTGACCAGGA AACCATATCAACCCTATGCTCCTGCTCGGGACTTTGCTGCTTACCGCCCACGCG CAAAGTTTTCTAGGAGCGCTGAAACCGCTGCCAACCTCCAAGACCCTAATCAGC TTTACAATGAATTGAACTTGGGACGCCGGGAGGAGTATGACGTCCTTGAGAAA AAGCGGGCTCGGGATCCAGAAATGGGCGGAAAGCAACAGAGGCGAAGAAATC CACAAGAGGGGGTCTATAACGCTCTTCAGAAAGATAAAATGGCTGAGGCATAT AGCGAAATTGGGACCAAGGGGGAGAGAAGAAGAGGCAAGGGACATGACGGGC TTTACCAGGGTTTGTCTACCGCAACAAAAGACACCTATGATGCTTTGCACATGC AAACACTGGCTCCTAGA-3′, (SEQ ID NO: 31) or an equivalent thereof.

### Host Cells and Processes for Preparing CARs

Aspects of the present disclosure relate to an isolated cell comprising a CAR and underexpressing a RelB polynucleotide, and methods of producing such cells. The cell is a prokaryotic or a eukaryotic cell. In one aspect, the cell is an immune cell, e.g., a T-cell, a B cell, a NK cell, a dendritic cell, a myeloid cell, a monocyte, a macrophage, any subsets thereof, or any other immune cell. The eukaryotic cell can be from any preferred species, e.g., an animal cell, a mammalian cell such as a human, a feline or a canine cell. The cells may be derived from patients, donors, or cell lines, such as those available off-the-shelf. The cells can be autologous or allogeneic to the subject being treated.

In specific embodiments, the isolated cell comprises, or alternatively consists essentially of, or yet further consists of an exogenous RelB and a CAR comprising, or alternatively consisting essentially of, or yet further consisting of, an antigen binding domain of a cancer or tumor antibody, a hinge domain, a transmembrane domain, one or more costimulatory signaling region, and optionally a CD3 zeta signaling domain. In certain embodiments, the isolated cell is a T-cell, e.g., an animal T-cell, a mammalian T-cell, a feline T-cell, a canine T-cell or a human T-cell. In certain embodiments, the isolated cell is an NK-cell, e.g., an animal NK-cell, a mammalian NK-cell, a feline NK-cell, a canine NK-cell or a human NK-cell. In certain embodiments, the isolated cell is a B-cell, e.g., an animal B-cell, a mammalian B-cell, a feline B-cell, a canine B-cell or a human B-cell. It is appreciated that the same or similar embodiments for each species apply with respect to dendritic cells, myeloid cells, monocytes, macrophages, any subsets of these or the T-cells, NK-cells, and B-cells described, and/or any other immune cells.

In certain embodiments, methods of producing the RelB and CAR expressing cells are disclosed, the method comprising, or alternatively consisting essentially of or yet further consisting of transducing a population of isolated cells with a nucleic acid sequence encoding the RelB and CAR. In a further aspect, a subpopulation of cells that have been successfully transduced with the nucleic acid sequences is selected. In some embodiments, the isolated cells are T-cells, an animal T-cell, a mammalian T-cell, a feline T-cell, a canine T-cell or a human T-cell, thereby producing the RelB and CAR. In certain embodiments, the isolated cell is an NK-cell, e.g., an animal NK-cell, a mammalian NK-cell, a feline NK-cell, a canine NK-cell or a human NK-cell, thereby producing the underexpressed RelB and CAR expressing immune cells. In some embodiments, the isolated cells are B-cells, an animal B-cell, a mammalian B-cell, a feline B-cell, a canine B-cell or a human B-cell, thereby underexpressing the RelB and CAR expressing B-cells. It is appreciated that the same or similar embodiments for each species apply with respect to dendritic cells, myeloid cells, monocytes, macrophages, any subsets of these or the T-cells, NK-cells, and B-cells described, and/or any other immune cells.

Sources of Isolated Cells. Prior to expansion and genetic modification of the cells disclosed herein, cells may be obtained from a subject—for instance, in embodiments involving autologous therapy—or a commercially available cell line or culture, or a stem cell such as an induced pluripotent stem cell (iPSC). In one aspect the subject is suffering from cancer. In another aspect, the subject is infected with a pathogen.

Cells can be obtained from a number of sources in a subject, including peripheral blood mononuclear cells, bone marrow, lymph node tissue, cord blood, thymus tissue, tissue from a site of infection, ascites, pleural effusion, spleen tissue, and tumors.

Methods of isolating relevant cells are well known in the art and can be readily adapted to the present application; an exemplary method is described in the examples below. Isolation methods for use in relation to this disclosure include but are not limited to Life Technologies Dynabeads® System; STEMcell Technologies EasySep™, RoboSep™, RosetteSep™, SepMate™; Miltenyi Biotec MACS™ cell separation kits, and other commercially available cell separation and isolation kits. Particular subpopulations of immune cells may be isolated through the use of beads or other binding agents available in such kits specific to unique cell surface markers. For example, MACS™ CD4+ and CD8+ MicroBeads may be used to isolate CD4+ and CD8+ T-cells. Alternative non-limiting examples of cells that may be isolated according to known techniques include bulked T-cells, NK T-cells, and gamma delta T-cells.

Alternatively, cells may be obtained through commercially available cell cultures, including but not limited to, for T-cells, lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™); for B cells, lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), and SUP-B15 (ATCC CRL-1929); and, for NK cells, lines NK-92 (ATCC® CRL-2407™), NK-92 MI (ATCC® CRL-2408™). Further examples include but are not limited to mature T-cell lines, e.g., Deglis, EBT-8, HPB-MLp-W, HUT 78, HUT 102, Karpas 384, Ki 225, My-La, Se-Ax, SKW-3, SMZ-1 and T34; immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); cutaneous T-cell lymphoma lines, e.g., HuT78 (ATCC CRM-TIB-161), MJ[G11] (ATCC CRL-8294), HuT102 (ATCC TIB-162); B-cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, and SU/RH-HD-1; and NK lines such as HANK1, KHYG-1, NKL, NK-YS, NOI-90, and YT. Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (atcc.org/) and the German Collection of Microorganisms and Cell Cultures (dsmz.de/).

In some embodiments, T-cells expressing the disclosed CARs may be further modified to reduce or eliminate expression of endogenous TCRs. Reduction or elimination of endogenous TCRs can reduce off-target effects and increase the effectiveness of the T cells. T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR. Even though some TCR complexes can be recycled to the cell surface when RNA interference is used, the RNA (e.g., shRNA, siRNA, miRNA, etc.) will prevent new production of TCR proteins resulting in degradation and removal of the entire TCR complex, resulting in the production of a T cell having a stable deficiency in functional TCR expression.

Expression of inhibitory RNAs (e.g., shRNA, siRNA, miRNA, etc.) in primary T cells can be achieved using any conventional expression system, e.g., a lentiviral expression system. Although lentiviruses are useful for targeting resting primary T cells, not all T cells will express the shRNAs. Some of these T cells may not express sufficient amounts of the RNAs to allow enough inhibition of TCR expression to alter the functional activity of the T cell. Thus, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3.

Expression of CRISPR in primary T cells can be achieved using conventional CRISPR/Cas systems and guide RNAs specific to the target TCRs. Suitable expression systems, e.g. lentiviral or adenoviral expression systems are known in the art. Similar to the delivery of inhibitor RNAs, the CRISPR system can be used to specifically target resting primary T cells or other suitable immune cells for CAR cell therapy. Further, to the extent that CRISPR editing is unsuccessful, cells can be selected for success according to the methods disclosed above. For example, as noted above, T cells that retain moderate to high TCR expression after viral transduction can be removed, e.g., by cell sorting or separation techniques, so that the remaining T cells are deficient in cell surface TCR or CD3, enabling the expansion of an isolated population of T cells deficient in expression of functional TCR or CD3. It is further appreciated that a CRISPR editing construct may be useful in both knocking out the endogenous TCR and knocking in the CAR constructs disclosed herein. Accordingly, it is appreciated that a CRISPR system can be designed for to accomplish one or both of these purposes.

Vectors. The immune cells can be prepared using vectors. Aspects of the present disclosure relate to an isolated nucleic acid sequence encoding (i) a CAR and (ii) an RelB encoding polynucleotide and a vector encoding (i) and a vector encoding (ii), and/or complements and/or equivalents of each thereof.

In some embodiments, the isolated nucleic acid sequence encodes for a CAR and comprises, or alternatively consists essentially of, or yet further consists of, a Kozak consensus sequence upstream of the sequence encoding the antigen binding domain of the cancer, tumor or pathogen targeting antibody.

In some embodiments, the isolated nucleic acid comprises a detectable label and/or a polynucleotide conferring antibiotic resistance. In one aspect, the label or polynucleotide are useful to select cells successfully transduced with the isolated nucleic acids.

In some embodiments, the isolated nucleic acid sequence is comprised within a vector. In certain embodiments, the vector is a plasmid. In other embodiments, the vector is a viral vector. Non-limiting examples of such include without limitation a retroviral vector, a lentiviral vector, an adenoviral vector, and an adeno-associated viral vector. In specific embodiments, the vector is a lentiviral vector.

The preparation of exemplary vectors and the generation of CAR and the RelB depleted cells using said vectors is discussed in detail in the examples below. In summary, the expression of natural or synthetic nucleic acids encoding CARs and the RelB is typically achieved by operably linking a nucleic acid encoding the polypeptide or portions thereof to a promoter, and incorporating the construct into an expression vector. The vectors can be suitable for replication and integration eukaryotes. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

In one aspect, the term “vector” intends a recombinant vector that retains the ability to infect and transduce non-dividing and/or slowly-dividing cells and integrate into the target cell's genome. In several aspects, the vector is derived from or based on a wild-type virus. In further aspects, the vector is derived from or based on a wild-type lentivirus. Examples of such include without limitation, human immunodeficiency virus (HIV), equine infectious anemia virus (EIAV), simian immunodeficiency virus (SIV) and feline immunodeficiency virus (FIV). Alternatively, it is contemplated that other retrovirus can be used as a basis for a vector backbone such murine leukemia virus (MLV). It will be evident that a viral vector according to the disclosure need not be confined to the components of a particular virus. The viral vector may comprise components derived from two or more different viruses and may also comprise synthetic components. Vector components can be manipulated to obtain desired characteristics, such as target cell specificity.

The recombinant vectors of this disclosure are derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699; 7,419,829 and 7,442,551, incorporated herein by reference.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′end of the RNA. The sizes of the three elements can vary considerably among different retroviruses. For the viral genome and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

Retroviral vectors for use in this disclosure include, but are not limited to Invitrogen's pLenti series versions 4, 6, and 6.2 “ViraPower” system. Manufactured by Lentigen Corp.; pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charité Medical School, Institute of Virology (CBF), Berlin, Germany.

Further methods of introducing exogenous nucleic acids into the art are known and include but are not limited to gene delivery using one or more of RNA electroporation, nanotechnology, sleeping beauty vectors, retroviruses, and/or adenoviruses.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Packaging vector and cell lines. The isolated nucleic acids can be packaged into a retroviral packaging system by using a packaging vector and cell lines. The packaging vector includes, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging vectors comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The retroviral packaging vector may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the packaging vectors and retroviral vectors are transiently cotransfected into a first population of mammalian cells that are capable of producing virus, such as human embryonic kidney cells, for example 293 cells (ATCC No. CRL1573, ATCC, Rockville, Md.) to produce high titer recombinant retrovirus-containing supernatants. In another method of the disclosure this transiently transfected first population of cells is then cocultivated with mammalian target cells, for example human lymphocytes, to transduce the target cells with the foreign gene at high efficiencies. In yet another method of the disclosure the supernatants from the above described transiently transfected first population of cells are incubated with mammalian target cells, for example human lymphocytes or hematopoietic stem cells, to transduce the target cells with the foreign gene at high efficiencies.

In another aspect, the packaging vectors are stably expressed in a first population of mammalian cells that are capable of producing virus, such as human embryonic kidney cells, for example 293 cells. Retroviral or lentiviral vectors are introduced into cells by either cotransfection with a selectable marker or infection with pseudotyped virus. In both cases, the vectors integrate. Alternatively, vectors can be introduced in an episomally maintained plasmid. High titer recombinant retrovirus-containing supernatants are produced.

Activation and Expansion of CAR Cells. Whether prior to or after genetic modification of the cells to express a desirable CAR, the cells can be activated and expanded using generally known methods such as those described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041 and references such as Lapateva et al. (2014) Crit Rev Oncog 19(1-2):121-32; Tam et al. (2003) Cytotherapy 5(3):259-72; Garcia-Marquez et al. (2014) Cytotherapy 16(11):1537-44. Stimulation with the tumor relevant antigen ex vivo can activate and expand the selected CAR expressing cell subpopulation. Alternatively, the cells can be activated in vivo by interaction with a tumor, cancer or pathogen-relevant antigen.

In the case of certain immune cells, additional cell populations, soluble ligands and/or cytokines, or stimulating agents may be required to activate and expand cells. The relevant reagents are well known in the art and are selected according to known immunological principles. For instance, soluble CD-40 ligand may be helpful in activating and expanding certain B-cell populations; similarly, irradiated feeder cells may be used in the procedure for activation and expansion of NK cells.

Methods of activating relevant cells are well known in the art and can be readily adapted to the present application; an exemplary method is described in the examples below. Isolation methods for use in relation to this disclosure include, but are not limited to Life Technologies Dynabeads® System activation and expansion kits; BD Biosciences Phosflow™ activation kits, Miltenyi Biotec MACS™ activation/expansion kits, and other commercially available cell kits specific to activation moieties of the relevant cell. Particular subpopulations of immune cells may be activated or expanded through the use of beads or other agents available in such kits. For example, α-CD3/α-CD28 Dynabeads® may be used to activate and expand a population of isolated T-cells.

Further provided is an immune cell prepared by the method described above. Also provided is a substantially homogenous population of cells as described herein. Also provided is a heterogeneous population of cells as described herein.

In one aspect, provided herein is an immune cell bound to the target cell.

### Compositions

Further provided are compositions comprising, or alternatively consisting essentially of, or yet further consisting of a carrier and one or more of any of the immune cell as described herein or the population of cells. In one aspect, the carrier is a pharmaceutically acceptable carrier. In a further aspect, the composition further comprises a cryoprotectant.

Briefly, pharmaceutical compositions of the present disclosure including but not limited to any one of the claimed compositions as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

### Kits

As set forth herein, the present disclosure provides methods for producing and administering immune cells. In one particular aspect, the present disclosure provides kits for performing these methods as well as instructions for carrying out the methods of the present disclosure such as collecting cells and/or tissues, and/or performing the screen/transduction/etc., and/or analyzing the results.

In one aspect the kit comprises, or alternatively consists essentially of, or yet further consists of, any one of the isolated nucleic acids disclosed herein and/or a vector comprising said nucleic acid and/or isolated allogenic cells, preferably T cells or NK cells, and/or instructions on the procuring of autologous cells from a patient. Such a kit may also comprise, or alternatively consist essentially of, or yet further comprise media and other reagents appropriate for the transduction and/or selection and/or activation and/or expansion of CAR and the RelB depleted cells, such as those disclosed herein.

In one aspect the kit comprises, or alternatively consists essentially of, or yet further consists of, an isolated CAR and the RelB depleted cells or population thereof. In some embodiments, the cells of this kit may require activation and/or expansion prior to administration to a subject in need thereof. In further embodiments, the kit may further comprise, or consist essentially thereof, media and reagents, such as those covered in the disclosure above, to activate and/or expand the isolated CAR and the RelB depleted cells. In some embodiments, the cell is to be used for CAR therapy. In further embodiments, the kit comprises instructions on the administration of the isolated cell to a patient in need of CAR therapy.

The kits of this disclosure can also comprise, e.g., a buffering agent, a preservative or a protein-stabilizing agent. The kits can further comprise components necessary for detecting the detectable-label, e.g., an enzyme or a substrate. The kits can also contain a control sample or a series of control samples, which can be assayed and compared to the test sample. Each component of a kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

### Therapeutic, Diagnostic and Screening Methods

The disclosure provides several therapeutic or screening methods. In one aspect, a method for inhibiting immune cell exhaustion in an immune cell expressing a CAR comprising, or consisting essentially of, or yet further consisting of depleteting in the immune cell a RelB polypeptide.

Also provided is one or methods for: rendering an immune cell less susceptible to exhaustion; enhancing the efficacy of CAR therapy; or reducing expression of Nra4a, Tox, PD-1, TIM3, LAG3, TIGIT and 2B4 in the immune cell expressing a CAR, the methods comprising, or consisting essentially of, or yet further consisting of underexpressing in the immune cell a RelB polypeptide.

Further provided is a method for stimulating a cell-mediated immune response comprising, or consisting essentially of, or yet further consisting of contacting a target cell population with the immune cell of this disclosure. The contacting can be in vitro or in vivo. In one aspect, the contacting is in vivo in a subject and the target cell population comprises cancer cells in the subject. In another aspect, the contacting is in vivo in a subject and the target cell population comprises pathogen infected cells in the subject. In one aspect, the immune cell specifically binds to a cell of the target population. The target cell can be a primary cell isolated from the subject or alternatively, it can be a cultured cell.

Alternatively, when the contacting is in vitro, the method is useful to screen for effective therapies, e.g., personalized therapies for the treatment of a specific patient or patient population.

When the target cell is a cancer cell, the subject has, has had or is in need of treatment for cancer or for a pathogenic infection the subject is infected with the pathogen.

As used herein, the expression and/or function of RelB is decreased as compared to a native immune cell or non-engineered cell. One can determine if RelB is decreased by detecting the level or amount of RelB mRNA or protein expressed by the cell using methods known in the art and described herein. One can also screen or assay for reduced expression of Nra4a, TOX, PD-1, TIM3, LAG3, TIGIT and 2B4 in the engineered cells as compared to cells without the RelB modification and/or increased expression of CD44, a marker of activated CD8 T cells; higher levels of the cytokines TNF and IFN-g after stimulation, and higher levels of several markers of effector CD8 T cells (KLRG1, granzyme B, CD107a).

The immune cell can be a primary immune cell or can be a cultured immune cell. Non-limiting examples of immune cells include, e.g., white blood cells (leukocytes, such as granulocytes (neutrophils, eosinophils, and basophils), macrophages, monocytes, and lymphocytes (T cells, B cells, natural killer (NK) cells and NKT cells)) which may be derived from hematopoietic stem cells (HSCs) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSCs), hematopoietic stem cells (HSCs), or immortalized cells. In some embodiments, the HSCs are derived from umbilical cord blood of a subject, peripheral blood of a subject, or bone marrow of a subject.

In one embodiment, the immune cell is from the group of a T cell, a CD4⁺ T cell, a CD8⁺ T cell, a macrophage, a stem cell or a Natural Killer (NK) T cell. In a particular aspect, the immune cell is a T cell, optionally a CD4⁺ T cell or a CD8⁺ T cell. The immune cell can be of any appropriate animal or mammalian species, e.g., canine, feline, equine, murine, rat or human

Also provided is a method for one or more of: promoting the survival and expansion of tumor-infiltrating immune cells such as CAR T cells; increasing the production of effector cytokines; decreasing the expression of inhibitory receptors and the exhaustion-associated transcription factor TOX; or generation of long-lived memory T cells that control tumor recurrence, in a subject in need thereof, the method comprising, or consisting essentially of, or yet further consisting of administering to the subject an effective amount of an immune cell as described herein. As is apparent, the tumor or cancer cell expresses an antigen for which the CAR is engineered to target.

Non-limiting examples of immune cells include, e.g., white blood cells (leukocytes, such as granulocytes (neutrophils, eosinophils, and basophils), macrophages, monocytes, and lymphocytes (T cells, B cells, natural killer (NK) cells and NKT cells)) which may be derived from hematopoietic stem cells (HSCs) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSCs), hematopoietic stem cells (HSCs), or immortalized cells. In some embodiments, the HSCs are derived from umbilical cord blood of a subject, peripheral blood of a subject, or bone marrow of a subject.

In one embodiment, the immune cell is from the group of a T cell, a CD4⁺ T cell, a CD8⁺ T cell, a macrophage, a stem cell or a Natural Killer (NK) T cell. In a particular aspect, the immune cell is a T cell, optionally a CD4⁺ T cell or a CD8⁺ T cell. The immune cell can be of any appropriate animal or mammalian species, e.g., canine, feline, equine, murine, rat or human. In addition, the subject can be an animal or mammal, e.g., canine, feline, equine, murine, rat or human.

In one embodiment, RelB function or expression is decreased by a vector expressing a polynucleotide encoding an siRNA that blocks the expression of RelB.

Also provided is a method of treating cancer in a subject, the method comprising, or consisting essentially of, or yet further consisting of administering to the subject the engineered immune cell as described herein. The cancer can be a liquid tumor or a solid tumor. In one aspect, the cancer expresses an antigen as disclosed herein, e.g., CD19, mesothelin, BMCA, ROR1, or EGFRvIII. In another aspect, the engineered immune cell selectively binds to the tumor antigen, e.g., an immune cell expressing an anti-BCMA antigen binding domain is administered to a subject having a cancer or tumor expressing BCMA. In another aspect, the engineered immune cell selectively binds to the tumor antigen, e.g., ROR1 and the immune cell expresses an anti-ROR1 antigen binding domain is administered to a subject having a cancer or tumor expressing ROR1. In another aspect, the engineered immune cell selectively binds to EGFRvIII and an immune cell expressing an anti-EGFRvIII antigen binding domain is administered to a subject having a cancer or tumor expressing EGFRvIII. In another aspect, the engineered immune cell selectively binds to mesothelin and an immune cell expressing an anti-mesothelin antigen binding domain is administered to a subject having a cancer or tumor expressing mesothelin.

Modes of administration are provided herein and include e.g., intravenous administration. The therapy can be first-line, second-line, third-line, fourth line, or fifth-line therapy.

Non-limiting examples of immune cells include, e.g., white blood cells (leukocytes, such as granulocytes (neutrophils, eosinophils, and basophils), macrophages, monocytes, and lymphocytes (T cells, B cells, natural killer (NK) cells and NKT cells)) which may be derived from hematopoietic stem cells (HSCs) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSCs), hematopoietic stem cells (HSCs), or immortalized cells. In some embodiments, the HSCs are derived from umbilical cord blood of a subject, peripheral blood of a subject, or bone marrow of a subject.

In one embodiment, the immune cell is from the group of a T cell, a CD4⁺ T cell, a CD8⁺ T cell, a macrophage, a stem cell or a Natural Killer (NK) T cell. In a particular aspect, the immune cell is a T cell, optionally a CD4⁺ T cell or a CD8⁺ T cell. The immune cell can be of any appropriate animal or mammalian species, e.g., canine, feline, equine, murine, rat or human. In addition, the subject can be an animal or mammal, e.g., canine, feline, equine, murine, rat or human.

Further provided is a method of providing anti-tumor immunity in a subject, the method comprising, or yet further consisting of administering to the subject the engineered immune cell as described herein. In one aspect, the subject's tumor or cancer cell expresses an antigen as disclosed herein and the immune cell is engineered to target the tumor cell.

The cancer can be a liquid tumor or a solid tumor. In one aspect, the cancer expresses an antigen as disclosed herein, e.g., CD19, mesothelin, BMCA, ROR1, or EGFRvIII. In another aspect, the engineered immune cell selectively binds to the tumor antigen, e.g., an immune cell expressing an anti-BCMA antigen binding domain is administered to a subject having a cancer or tumor expressing BCMA. In another aspect, the engineered immune cell selectively binds to the tumor antigen, e.g., ROR1 and the immune cell expresses an anti-ROR1 antigen binding domain is administered to a subject having a cancer or tumor expressing ROR1. In another aspect, the engineered immune cell selectively binds to EGFRvIII and an immune cell expressing an anti-EGFRVIII antigen binding domain is administered to a subject having a cancer or tumor expressing EGFRvIII. In another aspect, the engineered immune cell selectively binds to mesothelin and an immune cell expressing an anti-mesothelin antigen binding domain is administered to a subject having a cancer or tumor expressing mesothelin.

Modes of administration are provided herein and include e.g., intravenous administration. The therapy can be first-line, second-line, third-line, fourth line, or fifth-line therapy.

Non-limiting examples of immune cells include, e.g., white blood cells (leukocytes, such as granulocytes (neutrophils, eosinophils, and basophils), macrophages, monocytes, and lymphocytes (T cells, B cells, natural killer (NK) cells and NKT cells)) which may be derived from hematopoietic stem cells (HSCs) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSCs), hematopoietic stem cells (HSCs), or immortalized cells. In some embodiments, the HSCs are derived from umbilical cord blood of a subject, peripheral blood of a subject, or bone marrow of a subject.

In one embodiment, the immune cell is from the group of a T cell, a CD4⁺ T cell, a CD8⁺ T cell, a macrophage, a stem cell or a Natural Killer (NK) T cell. In a particular aspect, the immune cell is a T cell, optionally a CD4⁺ T cell or a CD8⁺ T cell. The immune cell can be of any appropriate animal or mammalian species, e.g., canine, feline, equine, murine, rat or human. In addition, the subject can be an animal or mammal, e.g., canine, feline, equine, murine, rat or human.

A method of treating a subject having a disease, disorder or condition associated with the expression of or an elevated expression of a tumor or cancer antigen, the method comprising, or yet further consisting of administering to the subject the engineered immune cell as described herein. In one aspect, the subject's tumor or cancer cell expresses an antigen as disclosed herein and the immune cell is engineered to target the tumor cell.

The cancer can be a liquid tumor or a solid tumor. In one aspect, the cancer expresses an antigen as disclosed herein, e.g., CD19, mesothelin, BMCA, ROR1, or EGFRvIII. In another aspect, the engineered immune cell selectively binds to the tumor antigen, e.g., an immune cell expressing an anti-BCMA antigen binding domain is administered to a subject having a cancer or tumor expressing BCMA. In another aspect, the engineered immune cell selectively binds to the tumor antigen, e.g., ROR1 and the immune cell expresses an anti-ROR1 antigen binding domain is administered to a subject having a cancer or tumor expressing ROR1. In another aspect, the engineered immune cell selectively binds to EGFRvIII and an immune cell expressing an anti-EGFRvIII antigen binding domain is administered to a subject having a cancer or tumor expressing EGFRvIII. In another aspect, the engineered immune cell selectively binds to mesothelin and an immune cell expressing an anti-mesothelin antigen binding domain is administered to a subject having a cancer or tumor expressing mesothelin.

Modes of administration are provided herein and include e.g., intravenous administration. The therapy can be first-line, second-line, third-line, fourth line, or fifth-line therapy.

Non-limiting examples of immune cells include, e.g., white blood cells (leukocytes, such as granulocytes (neutrophils, eosinophils, and basophils), macrophages, monocytes, and lymphocytes (T cells, B cells, natural killer (NK) cells and NKT cells)) which may be derived from hematopoietic stem cells (HSCs) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSCs), hematopoietic stem cells (HSCs), or immortalized cells. In some embodiments, the HSCs are derived from umbilical cord blood of a subject, peripheral blood of a subject, or bone marrow of a subject.

In one embodiment, the immune cell is from the group of a T cell, a CD4⁺ T cell, a CD8⁺ T cell, a macrophage, a stem cell or a Natural Killer (NK) T cell. In a particular aspect, the immune cell is a T cell, optionally a CD4⁺ T cell or a CD8⁺ T cell. The immune cell can be of any appropriate animal or mammalian species, e.g., canine, feline, equine, murine, rat or human. In addition, the subject can be an animal or mammal, e.g., canine, feline, equine, murine, rat or human.

A method of treating a pathogen infection in a subject, the method comprising, or yet further consisting of administering to the subject the engineered immune cell as described herein. In one aspect, the subject is infected with a pathogen that expresses a pathogenic antigen as disclosed herein and the immune cell is engineered to target the pathogenic antigen.

Modes of administration are provided herein and include e.g., intravenous administration.

Non-limiting examples of immune cells include, e.g., white blood cells (leukocytes, such as granulocytes (neutrophils, eosinophils, and basophils), macrophages, monocytes, and lymphocytes (T cells, B cells, natural killer (NK) cells and NKT cells)) which may be derived from hematopoietic stem cells (HSCs) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSCs), hematopoietic stem cells (HSCs), or immortalized cells. In some embodiments, the HSCs are derived from umbilical cord blood of a subject, peripheral blood of a subject, or bone marrow of a subject.

In one embodiment, the immune cell is from the group of a T cell, a CD4⁺ T cell, a CD8⁺ T cell, a macrophage, a stem cell or a Natural Killer (NK) T cell. In a particular aspect, the immune cell is a T cell, optionally a CD4⁺ T cell or a CD8⁺ T cell. The immune cell can be of any appropriate animal or mammalian species, e.g., canine, feline, equine, murine, rat or human. In addition, the subject can be an animal or mammal, e.g., canine, feline, equine, murine, rat or human.

A method of providing immunity to the pathogen infection in a subject, the method comprising, or yet further consisting of administering to the subject the engineered immune cell as described herein. In one aspect, the subject is infected with a pathogen that expresses a pathogenic antigen as disclosed herein and the immune cell is engineered to target the pathogenic antigen.

Modes of administration are provided herein and include e.g., intravenous administration.

Non-limiting examples of immune cells include, e.g., white blood cells (leukocytes, such as granulocytes (neutrophils, eosinophils, and basophils), macrophages, monocytes, and lymphocytes (T cells, B cells, natural killer (NK) cells and NKT cells)) which may be derived from hematopoietic stem cells (HSCs) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSCs), hematopoietic stem cells (HSCs), or immortalized cells. In some embodiments, the HSCs are derived from umbilical cord blood of a subject, peripheral blood of a subject, or bone marrow of a subject.

In one embodiment, the immune cell is from the group of a T cell, a CD4⁺ T cell, a CD8⁺ T cell, a macrophage, a stem cell or a Natural Killer (NK) T cell. In a particular aspect, the immune cell is a T cell, optionally a CD4⁺ T cell or a CD8⁺ T cell. The immune cell can be of any appropriate animal or mammalian species, e.g., canine, feline, equine, murine, rat or human. In addition, the subject can be an animal or mammal, e.g., canine, feline, equine, murine, rat or human.

A method for one or more of: inhibiting the growth of a tumor, killing a tumor, or inhibiting metastasis of a tumor in a cancer patient, comprising, or yet further consisting of administering to the subject the engineered immune cell as described herein. In one aspect, the subject's tumor cell expresses an antigen as disclosed herein and the immune cell is engineered to target the tumor cell. For example, the tumor cell expresses CD19 and the immune cell is engineered to target CD19, e.g., the immune cell expresses an anti-CD19 CAR.

The cancer can be a liquid tumor or a solid tumor. In one aspect, the cancer expresses an antigen as disclosed herein, e.g., CD19, mesothelin, BMCA, ROR1, or EGFRvIII. In another aspect, the engineered immune cell selectively binds to the tumor antigen, e.g., an immune cell expressing an anti-BCMA antigen binding domain is administered to a subject having a cancer or tumor expressing BCMA. In another aspect, the engineered immune cell selectively binds to the tumor antigen, e.g., ROR1 and the immune cell expresses an anti-ROR1 antigen binding domain is administered to a subject having a cancer or tumor expressing ROR1. In another aspect, the engineered immune cell selectively binds to EGFRvIII and an immune cell expressing an anti-EGFRVIII antigen binding domain is administered to a subject having a cancer or tumor expressing EGFRvIII. In another aspect, the engineered immune cell selectively binds to mesothelin and an immune cell expressing an anti-mesothelin antigen binding domain is administered to a subject having a cancer or tumor expressing mesothelin.

Modes of administration are provided herein and include e.g., intravenous administration. The therapy can be first-line, second-line, third-line, fourth line, or fifth-line therapy.

Non-limiting examples of immune cells include, e.g., white blood cells (leukocytes, such as granulocytes (neutrophils, eosinophils, and basophils), macrophages, monocytes, and lymphocytes (T cells, B cells, natural killer (NK) cells and NKT cells)) which may be derived from hematopoietic stem cells (HSCs) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSCs), hematopoietic stem cells (HSCs), or immortalized cells. In some embodiments, the HSCs are derived from umbilical cord blood of a subject, peripheral blood of a subject, or bone marrow of a subject.

In one embodiment, the immune cell is from the group of a T cell, a CD4⁺ T cell, a CD8⁺ T cell, a macrophage, a stem cell or a Natural Killer (NK) T cell. In a particular aspect, the immune cell is a T cell, optionally a CD4⁺ T cell or a CD8⁺ T cell. The immune cell can be of any appropriate animal or mammalian species, e.g., canine, feline, equine, murine, rat or human. In addition, the subject can be an animal or mammal, e.g., canine, feline, equine, murine, rat or human.

A method of treating a subject having a disease, disorder or condition associated with the expression of or an elevated expression of a tumor antigen, the method comprising, or yet further consisting of administering to the subject the engineered immune cell as described herein. In one aspect, the subject's tumor or cancer cell expresses an antigen as disclosed herein and the immune cell is engineered to target the tumor cell. For example, the tumor or cancer cell expresses CD19 and the immune cell is engineered to target CD19, e.g., the immune cell expresses an anti-CD19 CAR. The cancer can be a liquid tumor or a solid tumor. In one aspect, the cancer expresses an antigen as disclosed herein, e.g., CD19, mesothelin, BMCA, ROR1, or EGFRvIII. In another aspect, the engineered immune cell selectively binds to the tumor antigen, e.g., an immune cell expressing an anti-BCMA antigen binding domain is administered to a subject having a cancer or tumor expressing BCMA. In another aspect, the engineered immune cell selectively binds to the tumor antigen, e.g., ROR1 and the immune cell expresses an anti-ROR1 antigen binding domain is administered to a subject having a cancer or tumor expressing ROR1. In another aspect, the engineered immune cell selectively binds to EGFRvIII and an immune cell expressing an anti-EGFRvIII antigen binding domain is administered to a subject having a cancer or tumor expressing EGFRvIII. In another aspect, the engineered immune cell selectively binds to mesothelin and an immune cell expressing an anti-mesothelin antigen binding domain is administered to a subject having a cancer or tumor expressing mesothelin.

Modes of administration are provided herein and include e.g., intravenous administration. The therapy can be first-line, second-line, third-line, fourth line, or fifth-line therapy.

Non-limiting examples of immune cells include, e.g., white blood cells (leukocytes, such as granulocytes (neutrophils, eosinophils, and basophils), macrophages, monocytes, and lymphocytes (T cells, B cells, natural killer (NK) cells and NKT cells)) which may be derived from hematopoietic stem cells (HSCs) produced in the bone marrow, lymphocytes (T cells, B cells, natural killer (NK) cells, and NKT cells) and myeloid-derived cells (neutrophil, eosinophil, basophil, monocyte, macrophage, dendritic cells). In some embodiments, the immune cell is derived from one or more of the following: progenitor cells, embryonic stem cells, embryonic stem cell derived cells, embryonic germ cells, embryonic germ cell derived cells, stem cells, stem cell derived cells, pluripotent stem cells, induced pluripotent stem cells (iPSCs), hematopoietic stem cells (HSCs), or immortalized cells. In some embodiments, the HSCs are derived from umbilical cord blood of a subject, peripheral blood of a subject, or bone marrow of a subject.

In one embodiment, the immune cell is from the group of a T cell, a CD4⁺ T cell, a CD8⁺ T cell, a macrophage, a stem cell or a Natural Killer (NK) T cell. In a particular aspect, the immune cell is a T cell, optionally a CD4⁺ T cell or a CD8⁺ T cell. The immune cell can be of any appropriate animal or mammalian species, e.g., canine, feline, equine, murine, rat or human. In addition, the subject can be an animal or mammal, e.g., canine, feline, equine, murine, rat or human.

In the above methods, one can determine if the treatment by an endpoint as described herein. In one aspect, the methods provide one or more of promoting the survival and expansion of tumor-infiltrating CAR T cells; increasing the production of effector cytokines; decreasing the expression of inhibitory receptors and the exhaustion-associated transcription factor TOX; or generation of long-lived memory T cells that control tumor recurrence, in the subject.

In some embodiments, the isolated cell is autologous to the subject or patient being treated. In a further aspect, the tumor expresses a cancer or tumor antigen and the subject has been selected for the therapy by a diagnostic, such as use of an antibody that recognizes and binds the tumor or cancer relevant antigens targeted by the CARs. The subject is an animal, a mammal, a canine, a feline, a bovine, an equine, a murine or a human patient.

The engineered immune cells as disclosed herein may be administered either alone or in combination diluents, known anti-cancer therapeutics, and/or with other components such as cytokines or other cell populations that are immunoregulatory. They can be administered as a first line therapy, a second line therapy, a third line therapy, or further therapy. Non-limiting examples of additional therapies include cytoreductive therapy, such as radiation therapy, cryotherapy, or chemotherapy, or biologics. Further non-limiting examples include other relevant cell types, such as unmodified immune cells, modified immune cells comprising vectors expressing one or more immunoregulatory molecules, or CAR cells specific to a different antigen than those disclosed herein. As with the engineered immune cells of the present disclosure, in some embodiments, these cells may be autologous or allogenic. Appropriate treatment regimens will be determined by the treating physician or veterinarian.

Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials. In one aspect they are administered directly by direct injection or systemically such as intravenous injection.

Aspects of the disclosure provide an exemplary method for determining if a patient is likely to respond to, or is not likely to respond to, the engineered immune cells. The method comprises, or alternatively consists essentially thereof, or further consists of determining the presence or absence of a tumor associated antigen or a pathogenic antigen in a sample isolated from the patient and quantitating the amount of antigen or cells expressing the antigen. In certain embodiments, the method further comprises, or alternatively consists essentially of, or yet further consists of administering an effective amount of the engineered immune cells to the patient that is determined likely to respond to the engineered immune cells. The engineered immune cells can be autologous or allogenic to the patient and the patient can be subject that suffers from a solid tumor, animal or human.

Administration of the cells or compositions can be affected in one dose, continuously or intermittently throughout the course of treatment and an effective amount to achieve the desired therapeutic benefit is provided. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the disclosure can be administered in combination with other treatments.

The cells and populations of engineered immune cell are administered to the subject using methods known in the art and described, for example, in PCT/US2011/064191. This administration of the cells or compositions of the disclosure can be done to generate an animal model of the desired disease, disorder, or condition for experimental and screening assays.

Subjects suitable for the therapies includes but is not limited to a subject at risk of cancer or an infection, immune disorder, or autoimmune response, disorder or disease, as well as a subject that has already developed cancer or an age-associated genome dysfunction, immune disorder, or autoimmune response, disorder or disease. Such subjects, include mammalian animals (mammals), such as a non-human primate (apes, gibbons, gorillas, chimpanzees, orangutans, macaques), a domestic animal (dogs and cats), a farm animal (poultry such as chickens and ducks, horses, cows, goats, sheep, pigs), experimental animal (mouse, rat, rabbit, guinea pig) and humans. Subjects include animal disease models, for example, mouse and other animal models of cancer or an age-associated genome dysfunction, immune disorder, or autoimmune response, disorder or disease known in the art. In one aspect, the subject is an animal, mammal or human subject.

Accordingly, subjects appropriate for treatment include those having or at risk of cancer or an infection, immune disorder, or autoimmune response, disorder or disease, also referred to as subjects in need of treatment. Subjects in need of treatment therefore include subjects that have been previously had cancer or an infection, immune disorder, or autoimmune response, disorder or disease or that have an ongoing cancer or an infection, immune disorder, or autoimmune response, disorder or disease or have developed one or more adverse symptoms caused by or associated with cancer or an infection, immune disorder, or autoimmune response, disorder or disease, regardless of the type, timing or degree of onset, progression, severity, frequency, duration of the symptoms.

### Combination Therapies

The compositions as described herein can be administered as first line, second line, third line, fourth line, or other therapy and can be combined with cytoreductive interventions. The can be administered sequentially or concurrently as determined by the treating physician.

### EXPERIMENTAL EXAMPLES

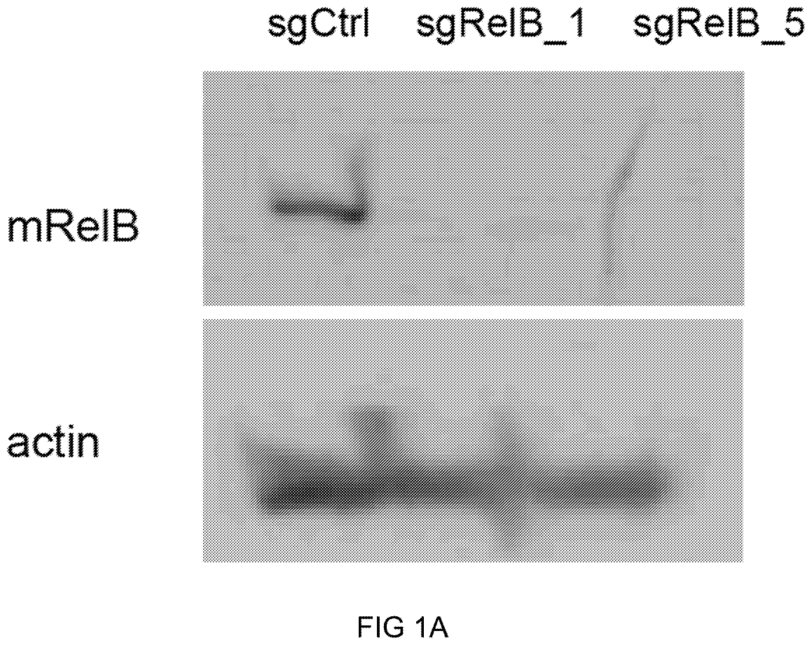

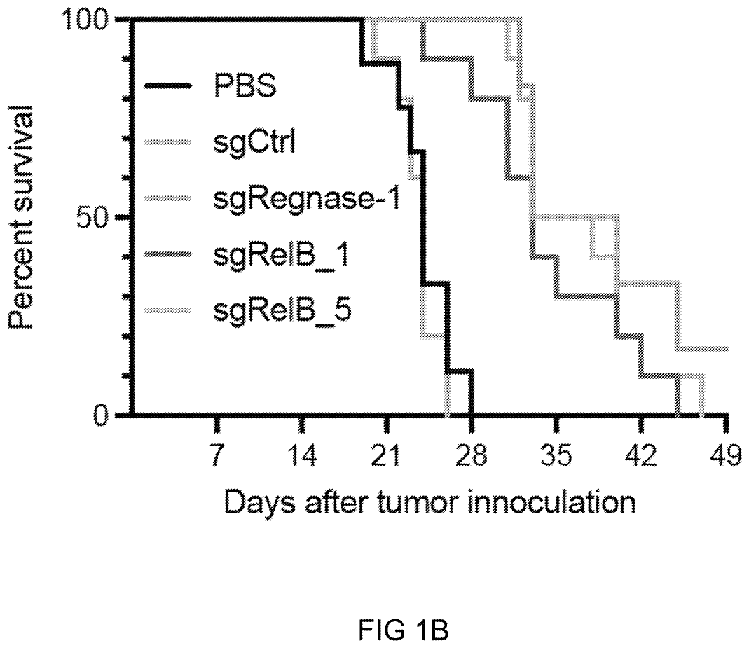

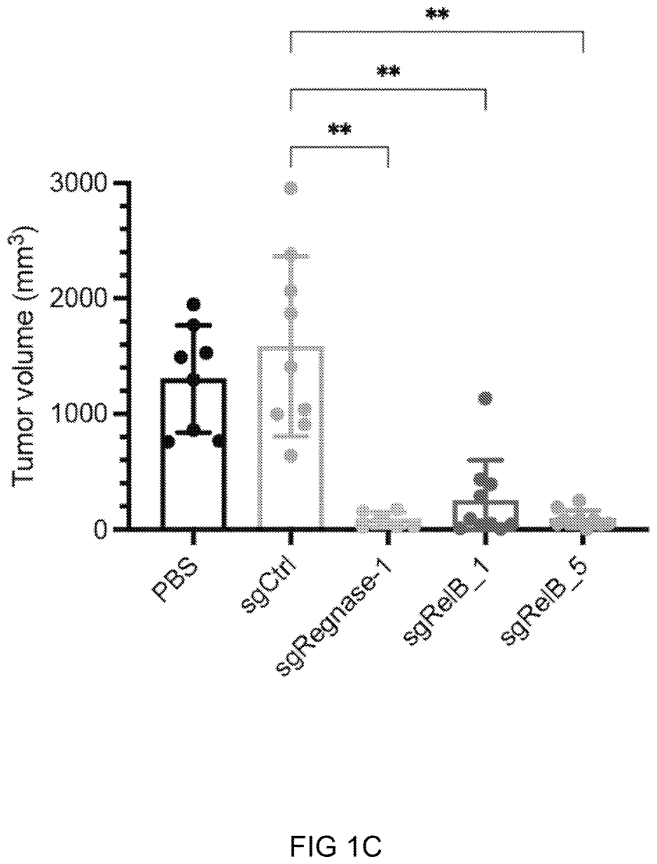

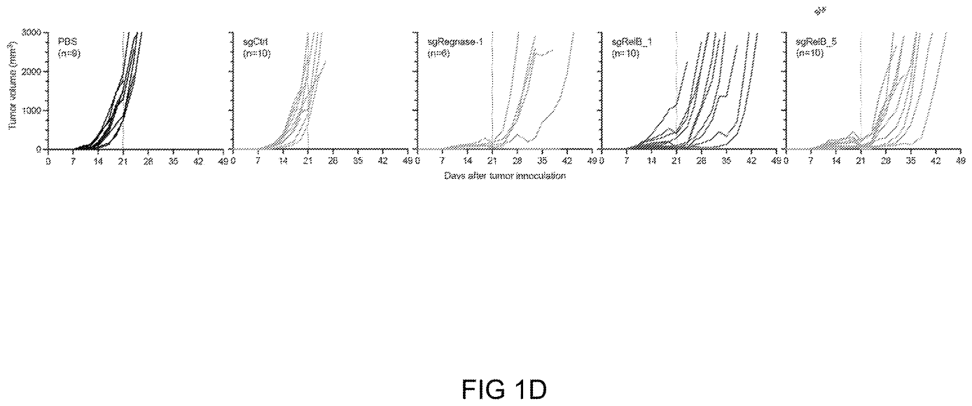

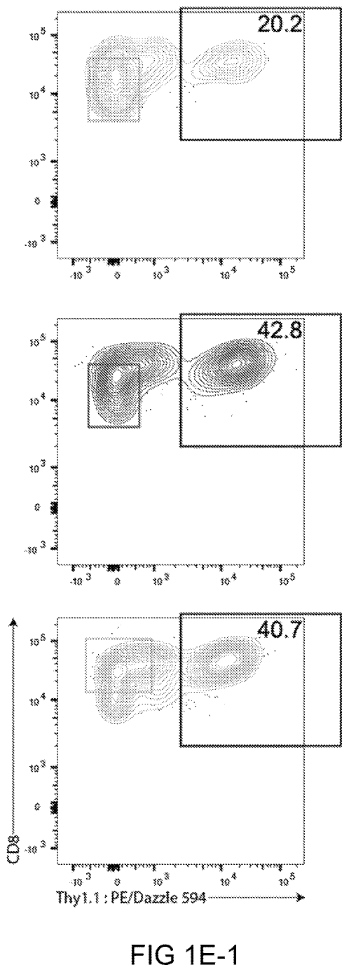

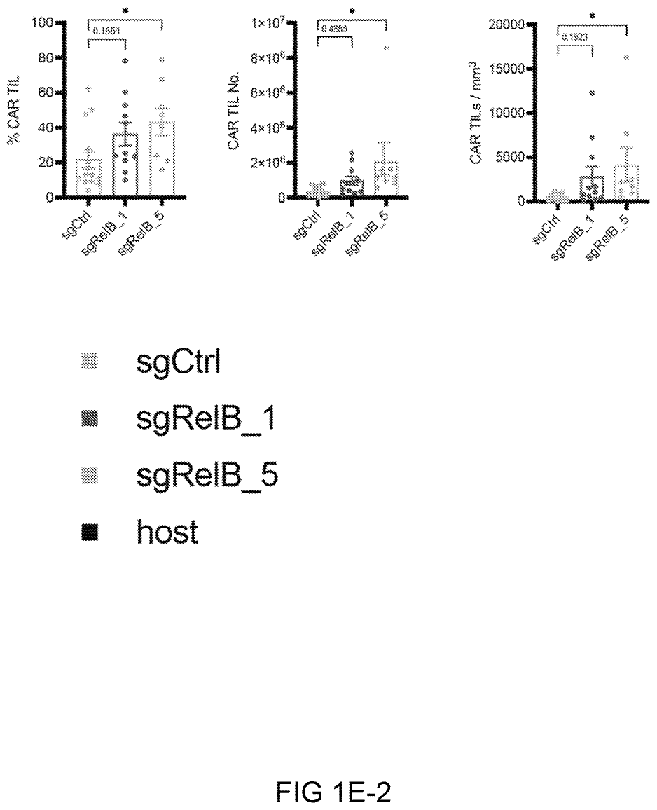

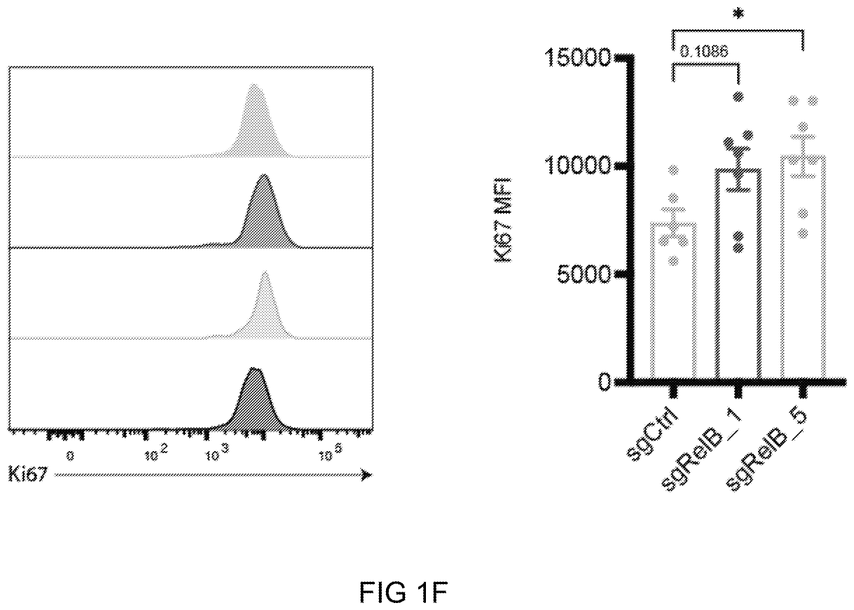

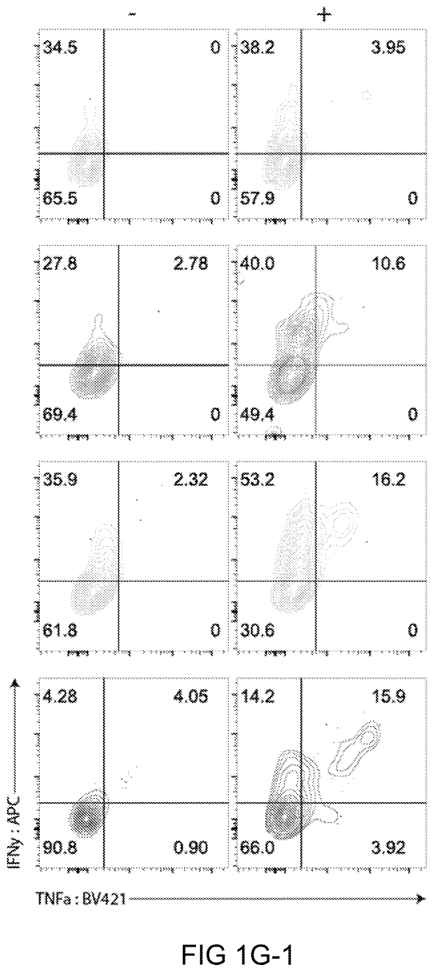

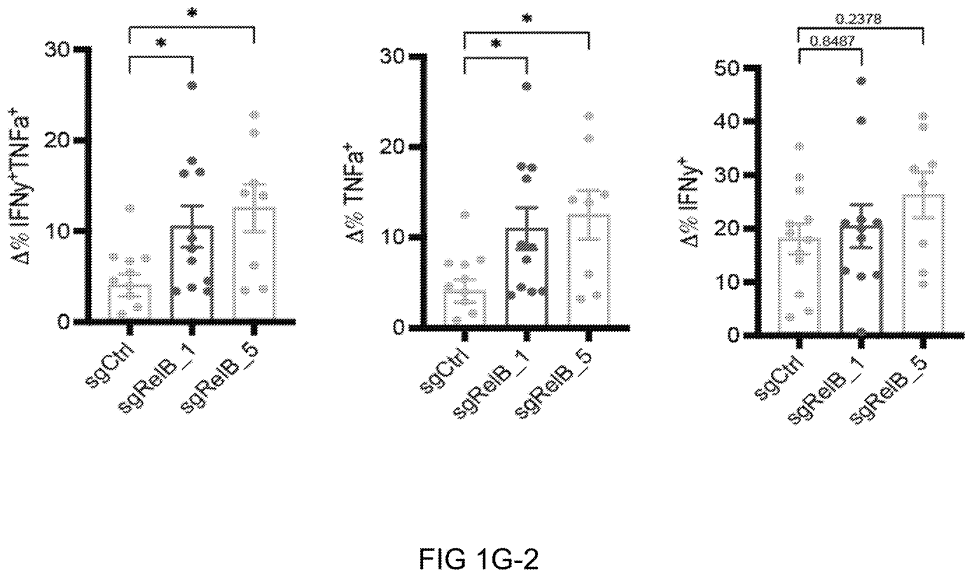

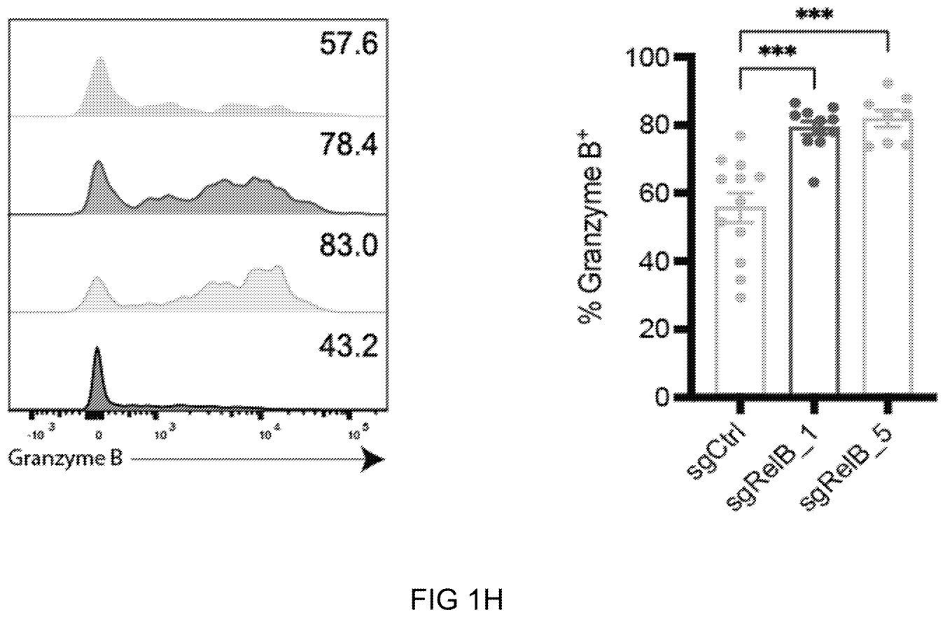

Using methods similar to those disclosed in Applicant's copending published application, US 2020/0352999, published Nov. 12, 2020. In FIGS. 1A-H, Applicant has demonstrated that adoptively transferring tumor-bearing mice with RelB-depleted mouse CD8 CAR-T cells improves survival and significantly delays tumor progression (See FIGS. 1, A-D). RelB-depletion also improves CAR-T cell expansion as shown by the higher frequency, absolute number, and density of tumor infiltrating lymphocytes (See FIG. 1E), as well as higher levels of Ki-67 (FIG. 1F). Furthermore, Rel-B depletion improved production of the cytokine TNFa upon restimulation (FIG. 1G), and increased levels of Granzyme B under resting conditions (FIG. 1H).

Further, Applicant has tested if RelB knockdown can improve human CAR-T cell efficacy in two different models: a liquid NALM6 model with anti-human CD19-CAR, and a solid tumor model (AsPC-1) with anti-human mesothelin CAR. RelB knockdown in these models improves survival and delays tumor progression.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a/proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, GenBank citations, ATCC citations, and other references mentioned herein are expressly incorporated by reference in their entirety, to the same extent as if each were incorporated by reference individually. In case of conflict, the specification, including definitions, will control.

Other aspects are set forth within the following claims.

1. An immune cell engineered to decrease one or more of:
a) expression and/or function of an isoform of RelB in the immune cell;
b) expression and/or function of T cell exhaustion proteins in the immune cell; and/or
c) expression and/or function of an isoform of RelB and to decrease expression and/or function of T cell exhaustion proteins in the immune cell.

2. The immune cell of claim 1, wherein the T cell exhaustion protein is Nra4a or Tox.

3. The immune cell of claim 1, wherein the immune cell expresses a receptor or ligand that binds at least one tumor antigen or at least one antigen expressed by a pathogen, optionally wherein the antigen is a tumor antigen selected from the group of CD19, mesothelin, ROR1, or EGFRVIII.

4.-5. (canceled)

6. The immune cell of claim 1, wherein the immune cell is a CD4⁺ T cell or a CD8⁺ T cell.

7. (canceled)

8. The immune cell of claim 3, wherein the immune cell comprises:
a) a chimeric antigen receptor (CAR) comprising (a) an antigen binding domain; (b) a hinge domain; (c) a transmembrane domain; (d) and an intracellular domain, and optionally wherein the antigen binding domain comprises the receptor or ligand, or
b) a polynucleotide encoding the CAR.

9.-10. (canceled)

11. The immune cell of claim 1, wherein the immune cell under-expresses the isoform of RelB in the immune cell as compared to a naturally occurring immune cell.

12. The immune cell of claim 8, wherein the antigen binding domain of the CAR comprises a single-chain variable fragment (scFv) of an antigen binding domain of a humanized antibody, and wherein the antigen binding domain comprises: an anti-CD19 binding domain scFv of an anti-CD19 antibody; a heavy chain variable region and a light chain variable region of an anti-CD19 antibody; or the 6 complementarity-determining regions (CDRs) of an anti-CD19 antibody.

13.-20. (canceled)

21. The immune cell of claim 8 wherein the antigen binding domain comprises the antigen binding domain of the group of: anti-mesothelin antibody, an anti-ROR1 antibody, or an anti-EGFRvIII antibody.

22. (canceled)

23. The immune cell of claim 1, wherein the immune cell has been isolated from a subject and is subsequently modified to decrease expression of RelB.

24. The immune cell of claim 23, wherein the subject has cancer, and a tumor antigen is expressed by a cell associated with the cancer, or
wherein the subject is infected with a pathogen, and wherein the cell expresses an antigen associated with infection.

25.-26. (canceled)

27. A method of producing an engineered immune cell, the method comprising decreasing expression and/or function of an isoform of RelB in the immune cell.

28.-54. (canceled)

55. An immune cell prepared by the method of claim 27.

56. A substantially homogenous population of cells claim 1.

57. (canceled)

58. A composition comprising a carrier and the cell of claim 1.

59.-61. (canceled)

62. A kit comprising vectors and instructions for the manufacture of the cell of claim 1.

63. A method for stimulating a cell-mediated immune response, the method comprising contacting a target cell population with the cell of claim 1.

64.-68. (canceled)

69. A method of treating cancer or providing anti-tumor immunity in a subject, the method comprising administering to the subject the cell of claim 1.

70.-72. (canceled)

73. A method of treating a subject having a disease, disorder or condition associated with the expression of or an elevated expression of a tumor antigen, the method comprising administering to the subject the cell of claim 1.

74. A method of treating a pathogen infection in a subject, or providing immunity to the pathogen infection in a subject, the method comprising administering to the subject the cell of claim 1.

75. (canceled)

76. A method for one or more of: inhibiting the growth of a tumor, killing a tumor, or inhibiting metastasis of a tumor in a cancer patient, comprising administering to the subject the cell of claim 1.

77.-86. (canceled)


SEQUENCE LISTING (38 sequences)

SEQ ID NO: 1 - protein, 579 aa, Homo sapiens
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 2 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 229 aa, Homo sapiens; identical to SEQ ID NO: 2

SEQ ID NO: 4-6 - skipped (3 entries)

SEQ ID NO: 7 - DNA, 48 bp, synthetic construct
  source 1..48: mol_type = other DNA
ctcgagcccaaatcttgtgacaaaactcacacatgcccaccgtgcccg

SEQ ID NO: 8 - DNA, 81 bp, Homo sapiens
  source 1..81: mol_type = genomic DNA
ttttgggtgctggtggtggttggtggagtcctggcttgctatagcttgctagtaacagtggcctttattattttctgggtg

SEQ ID NO: 9 - DNA, 126 bp, Homo sapiens
  source 1..126: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 10 - DNA, 123 bp, Homo sapiens
  source 1..123: mol_type = genomic DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 11 - DNA, 339 bp, Homo sapiens
  source 1..339: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 12 - protein, 51 aa, Homo sapiens
PAKPTTTPAPRPPTPAPTIASQPLSLRPEACRPAAGGAVHTRGLDFACDIY

SEQ ID NO: 13 - protein, 49 aa, Mus musculus
KVNSTTTKPVLRTPSPVHPTGTSQPQRPEDCRPRGSVKGTGLDFACDIY

SEQ ID NO: 14 - protein, 51 aa, Felis sp.
PVKPTTTPAPRPPTQAPITTSQRVSLRPGTCQPSAGSTVEASGLDLSCDIY

SEQ ID NO: 15 - protein, 21 aa, Homo sapiens
IYIWAPLAGTCGVLLLSLVIT

SEQ ID NO: 16 - protein, 21 aa, Mus musculus
IWAPLAGICVALLLSLIITLI

SEQ ID NO: 17 - protein, 21 aa, Rattus norvegicus
IWAPLAGICAVLLLSLVITLI

SEQ ID NO: 18 - skipped

SEQ ID NO: 19 - DNA, 81 bp, Homo sapiens; identical to SEQ ID NO: 8
  source 1..81: mol_type = genomic DNA

SEQ ID NO: 20 - protein, 27 aa, Homo sapiens
FWVLVVVGGVLACYSLLVTVAFIIFWV

SEQ ID NO: 21 - DNA, 126 bp, Homo sapiens; identical to SEQ ID NO: 9
  source 1..126: mol_type = genomic DNA

SEQ ID NO: 22 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 23 - DNA, 123 bp, Homo sapiens; identical to SEQ ID NO: 10
  source 1..123: mol_type = genomic DNA

SEQ ID NO: 24 - DNA, 105 bp, Homo sapiens
  source 1..105: mol_type = genomic DNA
acaaaaaagaagtattcatccagtgtgcacgaccctaacggtgaatacatgttcatgagagcagtgaacacagccaaaaaatccagactcacagatgtgacccta

SEQ ID NO: 25 - DNA, 108 bp, Homo sapiens
  source 1..108: mol_type = genomic DNA
agggaccagaggctgccccccgatgcccacaagccccctgggggaggcagtttccggacccccatccaagaggagcaggccgacgcccactccaccctggccaagatc

SEQ ID NO: 26 - protein, 20 aa, Homo sapiens
RPRRSPAQDGKVYINMPGRG

SEQ ID NO: 27 - protein, 21 aa, Homo sapiens
ESPYQELQGQRSDVYSDLNTQ

SEQ ID NO: 28 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 339 bp, Homo sapiens; identical to SEQ ID NO: 11
  source 1..339: mol_type = genomic DNA

SEQ ID NO: 30 - protein, 490 aa, synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 31 - DNA, 1470 bp, synthetic construct
  source 1..1470: mol_type = other DNA
atggctttgccagtgacagctcttctccttccactggccctcctccttcacgccgctaggccagagcagaaacttatttcagaggaagacctggacattcaaatgacacaaactacttcttctctctccgcctcacttggtgaccgcgtcactattagttgccgcgctagtcaagatattagtaagtacctgaattggtatcaacaaaaacctgacgggactgtaaagctgcttatatatcatacttctaggctgcattctggagtaccttcacgatttagcggtagcggatccggcaccgactactccctcacaattagcaatctggagcaagaggacatagccacctacttctgccagcaagggaataccttgccatacactttcggtggtggaactaagctcgaaattactgggggtggaggcagtggcggaggggggtcaggtgggggaggttcagaagtcaaactccaggaatctggacctggactcgttgccccttcccaatcccttagtgttacatgcactgtatcaggtgtatccctccctgattacggtgtctcctggattcggcagcctcctcggaagggtctcgagtggttgggagtgatttgggggtctgaaactacttattataacagtgcccttaagagtagattgactataattaaggataacagtaagtcacaagtattcctcaaaatgaattccttgcaaacagacgatacagcaatatattactgcgcaaaacactactactatggcggtagttacgctatggactattggggtcaaggaacctctgtcacagtttctagcattgagttcatgtatcccccaccttacttggacaatgaaaggtctaatgggaccatcatacacattaaagagaaacacctgtgtcatactcagagttctccaaaattgttctgggccttggttgtcgttgccggcgtactgttctgttacggtctcttggttaccgtggcactttgtgttatctggactaattcccggcggaatcggggtggacagagcgattacatgaatatgaccccaagaagacctggactgaccaggaaaccatatcaaccctatgctcctgctcgggactttgctgcttaccgcccacgcgcaaagttttctaggagcgctgaaaccgctgccaacctccaagaccctaatcagctttacaatgaattgaacttgggacgccgggaggagtatgacgtccttgagaaaaagcgggctcgggatccagaaatgggcggaaagcaacagaggcgaagaaatccacaagagggggtctataacgctcttcagaaagataaaatggctgaggcatatagcgaaattgggaccaagggggagagaagaagaggcaagggacatgacgggctttaccagggtttgtctaccgcaacaaaagacacctatgatgctttgcacatgcaaacactggctcctaga

SEQ ID NO: 32 - protein, 58 aa, Homo sapiens
MLRSGPASGPSVPTGRAMPSRRVARPPAAPELGALGSPDLSSLSLAVSRSTGSSSTDA

SEQ ID NO: 33 - protein, 576 aa, Homo sapiens
MLRSGPASGPSVPTGRAMPSRRVARPPAAPELGALGSPDLSSLSLAVSRSTEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 34 - protein, 115 aa, Homo sapiens
EIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIEF

SEQ ID NO: 35 - protein, 142 aa, synthetic construct
  VARIANT 1: note = Any amino acid
XSEPLPFTYLPRDHGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 36 - protein, 42 aa, Homo sapiens
KRGRKKLLYIFKQPFMRPVQTTQEEDGCSCRFPEEEEGGCEL

SEQ ID NO: 37 - protein, 41 aa, Homo sapiens
RSKRSRLLHSDYMNMTPRRPGPTRKHYQPYAPPRDFAAYRS

SEQ ID NO: 38 - protein, 30 aa, synthetic construct
  VARIANT 1..30: note = This sequence may encompass 1-6 GGGGS repeating units
GGGGSGGGGSGGGGSGGGGSGGGGSGGGGS